SEQUENCE LISTING (50 sequences)

SEQ ID NO: 1 - protein, 10 aa, Artificial Sequence
  Peptide
MDWWPMWPSL

SEQ ID NO: 2 - protein, 10 aa, Artificial Sequence
  Peptide
MWMIQMPGYQ

SEQ ID NO: 3 - protein, 10 aa, Artificial Sequence
  Peptide
MRWSMSYSWA

SEQ ID NO: 4 - protein, 10 aa, Artificial Sequence
  Peptide
MFMMMWMSLT

SEQ ID NO: 5 - protein, 10 aa, Artificial Sequence
  Peptide
MFFVLSWTPL

SEQ ID NO: 6 - protein, 10 aa, Artificial Sequence
  Peptide
MQMWTMWEPW

SEQ ID NO: 7 - protein, 10 aa, Artificial Sequence
  Peptide
MDLWPMWESW

SEQ ID NO: 8 - protein, 10 aa, Artificial Sequence
  Peptide
MWQMMNGMSQ

SEQ ID NO: 9-12 - skipped (4 entries)

SEQ ID NO: 13 - protein, 10 aa, Artificial Sequence
  Peptide
MAWWPMWPSL

SEQ ID NO: 14 - protein, 10 aa, Artificial Sequence
  Peptide
MDAWPMWPSL

SEQ ID NO: 15 - protein, 10 aa, Artificial Sequence
  Peptide
MDWWAMWPSL

SEQ ID NO: 16 - protein, 10 aa, Artificial Sequence
  Peptide
MDWWPAWPSL

SEQ ID NO: 17 - protein, 10 aa, Artificial Sequence
  Peptide
MDWWPMWASL

SEQ ID NO: 18 - protein, 10 aa, Artificial Sequence
  Peptide
MDWWPMWPAL

SEQ ID NO: 19 - protein, 10 aa, Artificial Sequence
  Peptide
MDWWPMWPSA

SEQ ID NO: 20 - protein, 10 aa, Artificial Sequence
  Peptide
MDWWPLWPSL

SEQ ID NO: 21 - protein, 10 aa, Artificial Sequence
  Peptide
MDWWPTWPSL

SEQ ID NO: 22 - protein, 10 aa, Artificial Sequence
  Peptide
MDWWPVWPSL

SEQ ID NO: 23 - protein, 11 aa, Artificial Sequence
  Peptide
MMDWWPMWPSL

SEQ ID NO: 24 - protein, 11 aa, Artificial Sequence
  Peptide
MLDWWPMWPSL

SEQ ID NO: 25 - protein, 11 aa, Artificial Sequence
  Peptide
MMDWWPLWPSL

SEQ ID NO: 26 - protein, 11 aa, Artificial Sequence
  Peptide
MLDWWPLWPSL

SEQ ID NO: 27 - protein, 10 aa, Artificial Sequence
  Peptide
MAQMMNGMSQ

SEQ ID NO: 28 - protein, 10 aa, Artificial Sequence
  Peptide
MWAMMNGMSQ

SEQ ID NO: 29 - protein, 10 aa, Artificial Sequence
  Peptide
MWQAMNGMSQ

SEQ ID NO: 30 - protein, 10 aa, Artificial Sequence
  Peptide
MWQMANGMSQ

SEQ ID NO: 31 - protein, 10 aa, Artificial Sequence
  Peptide
MWQMMAGMSQ

SEQ ID NO: 32 - protein, 10 aa, Artificial Sequence
  Peptide
MWQMMAGMSQ

SEQ ID NO: 33 - protein, 10 aa, Artificial Sequence
  Peptide
MWQMMNGASQ

SEQ ID NO: 34 - protein, 10 aa, Artificial Sequence
  Peptide
MWQMMNGMAQ

SEQ ID NO: 35 - protein, 10 aa, Artificial Sequence
  Peptide
MWQMMNGMAQ

SEQ ID NO: 36 - protein, 8 aa, Artificial Sequence
  Peptide
ASGTSGSS

SEQ ID NO: 37 - protein, 8 aa, Artificial Sequence
  Peptide
GAGTSGSS

SEQ ID NO: 38 - protein, 8 aa, Artificial Sequence
  Peptide
GSATSGSS

SEQ ID NO: 39 - protein, 18 aa, Artificial Sequence
  Peptide
MWQMMNGMSQGSGTSGSS

SEQ ID NO: 40 - protein, 11 aa, Artificial Sequence
  Peptide
MWQMMNGMSQA

SEQ ID NO: 41 - protein, 11 aa, Artificial Sequence
  Peptide
MWQMMNGMSQG

SEQ ID NO: 42 - protein, 12 aa, Artificial Sequence
  Peptide
MWQMMNGMSQGS

SEQ ID NO: 43 - protein, 18 aa, Artificial Sequence
  Peptide
MWQMMNGMSQASGTSGSS

SEQ ID NO: 44 - protein, 18 aa, Artificial Sequence
  Peptide
MWQMMNGMSQGAGTSGSS

SEQ ID NO: 45 - protein, 19 aa, Artificial Sequence
  Peptide
MMWQMMNGMSQGSGTSGSS

SEQ ID NO: 46 - protein, 19 aa, Artificial Sequence
  Peptide
MAWQMMNGMSQGSGTSGSS

SEQ ID NO: 47 - protein, 4 aa, Artificial Sequence
  Peptide
GSGT

SEQ ID NO: 48 - protein, 5 aa, Artificial Sequence
  Peptide
GSGTS

SEQ ID NO: 49 - protein, 7 aa, Artificial Sequence
  Peptide
MMNGMSQ

SEQ ID NO: 50 - protein, 10 aa, Artificial Sequence
  Peptide
GSGTSGSSGS

(19) United States
(12) Patent Application Publication
(10) Pub. No.: US 2023/0322854 A1
(43) Pub. Date: Oct. 12, 2023

(54) HUNTINGTIN-RELATED PEPTIDE AGENTS AND USES THEREOF

(71) Applicant: UNIVERSITY OF SOUTHERN CALIFORNIA, LOS ANGELES, CA (US)

(72) Inventors: Richard W. ROBERTS, Los Angeles, CA (US); Ralf LANGEN, Los Angeles, CA (US); Jeannie CHEN, Los Angeles, CA (US); Jose Mario ISAS, Los Angeles, CA (US); Kaori NORIDOMI, Los Angeles, CA (US); Terry TAKAHASHI, Los Angeles, CA (US)

(21) Appl. No.: 18/044,028

(22) PCT Filed: Sep. 3, 2021
(86) PCT No.: PCT/US2021/049154

§ 371 Date: Mar. 3, 2023

Related U.S. Application Data
(60) Provisional application No. 63/074,250, filed on Sep. 3, 2020.

(51) Int. Cl. C07K 7/06; G01N 33/68; A61P 25/14

(52) U.S. Cl. CPC C07K 7/06; G01N 33/6896; A61P 25/14; G01N 2800/2835; G01N 2800/50

(57) ABSTRACT

Presented herein are novel peptides and peptide agents for diagnosing, preventing or treating Huntingtin's Disease.

### CROSS REFERENCE TO RELATED APPLICATION

This application claims priority to U.S. Provisional Application Serial No. 63/074,250, filed Sep. 3, 2020, which is hereby incorporated by reference in its entirety.

Embodiments relate to peptide agents, and uses thereof.

### INTRODUCTION

Huntington's disease (HD) is an autosomal dominant neurodegenerative disease. HD is thought to be caused by the aggregation of mutant Huntingtin protein that contain expanded polyglutamine (PolyQ) repeats in exon 1. PolyQ length of Huntingtin is inversely correlated with age of disease onset, with longer PolyQ lengths resulting in earlier onset of disease (Walker (2007) Lancet 369: 218-28). The aggregation of Huntingtin often occurs with PolyQ lengths >36, and occurs by the association of monomeric Huntingtin into an alpha-helical rich oligomer, which can then assemble into an unbundled fibril, and the unbundled fibrils assembling into bundled fibrils (FIG. 1). The N-terminal region of Huntingtin, including the first 17 residues (N17), the polyQ region (PolyQ), and a proline-rich domain (PRD) are sufficient for formation of fibrils (FIG. 1) and are a model system for studying Huntingtin fibril formation. The aggregation of Huntingtin is hypothesized to be causative of HD.

### SUMMARY

Presented herein, in certain aspects, are compositions, peptide agents and peptides that can inhibit Huntingtin aggregation. In some embodiments, the compositions, peptide agents and/or peptides disclosed herein can be used to treat Huntington's disease (HD), to prevent or delay the onset or progression of HD, to reduce the severity of one or more symptoms of HD and/or to diagnose HD.

In some aspects, presented herein is a method of treating HD in a subject in need thereof comprising administering a therapeutically effective amount of a composition, peptide agent or peptide disclosed herein to the subject.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows an example of a Huntingtin aggregation pathway. Panel A shows a schematic of the N17, Polyglutamine rich region (PolyQ), and the Proline rich domain (PRD) of exon 1. Panel B shows hypothesized aggregation pathway starting with monomers that aggregate into a soluble, early oligomer, which then further aggregates into an unbundled fibril. These fibrils can further associate into a bundled fibril.

FIG. 2 shows an mRNA display process.

FIG. 3 is a bar graph showing binding of a peptide library against unbundled Huntingtin (Htt) fibrils.

FIG. 4 shows clones identified by DNA sequencing of mRNA display libraries selected against unbundled fibrils of Huntingtin Exon 1.

FIG. 5 is a bar graph showing clone binding vs. Fibril or monomeric Huntingtin (Monomer). Pool 6 is shown as a comparison with Clones 1 through 5 (HD1 through HD5, respectively). This comparison was realized using 20 μL Beads Slurry in 1 mL buffer, 2 μg protein (about 140 pmol as monomer).

FIG. 6 shows clone binding to Htt-Fibrils. Pool 6 and Clone 1 (HD1) are shown as a comparison. Clone 6-8 (HD6 through HD8, respectively) were tested in this assay using 20 μL Beads Slurry in 1 mL buffer, 4 μg protein (˜280 pmol as monomer).

FIG. 7 shows characterization of HD1 and HD8 binding. The left panel shows a comparison of HD clones versus QBP1, in which HD1 and HD8 were tested versus QPB1, another peptide identified to bind polyQ protein aggregates. The right panel shows testing of HD1 and HD8 binding versus α-synuclein fibrils, which demonstrated binding specificity for Huntingtin fibrils. On the left panel, from left to right and for each dataset (i.e., “HD1”, “HD8” and “QBP1”), the bars represent “No Target”, “3.2 Fibril”, and “Monomer”. On the right panel, from left to right and for each dataset (i.e., “HD1 and “HD8”), the bars represent “No Target”, “3.2 Fibril”, “Monomer”, and “a-Synuclein”.

FIG. 8 shows testing competition with chemically synthesized HD1 and HD8. Radiolabeled HD1 or HD8 were bound to immobilized fibrils and the appropriate compound was tested in a competition binding assay. From left to right and for each dataset shown on the graph (i.e., “HD1” and “HD8”), the bars represent “No Target+DMSO”, “No Target+HD1”, “No Target+HD8”, “3.2 Fibril+DMSO”, “3.2 Fibril+HD1”, and “3.2 Fibril+HD8”.

FIG. 9 presents electron micrography of fibril formation over 6 days using a control (no peptide added; left panels) or addition of HD1 or HD8 (right panels), which showed significant inhibition of fibril formation.

FIG. 10 presents graphs showing testing binding of chemically synthesized HD1 or HD8 using Electron Paramagnetic Resonance (EPR). Panel A shows EPR spectra of EPR spectra of spin-labeled peptide in the absence (“HD 1” and “HD 8” curves) or presence of fibrils (“HD 1+Fibril” and “HD 8+Fibril” curves). A decrease in central line amplitude indicated the formation of a complex. Panel B is a graph showing the results of experiments aimed at the determination of the stoichiometry of peptides binding to Httex1 molecules in the fibril, in which a spin-labeled peptide (HD1 or HD8) is added to 15 μM fibrils. HD1 peptide binding was saturated at a 1 to 3 ratio whereas HD8 showed a linear response through equal concentrations of peptide and fibril. These results indicated that the HD1 and HD8 peptides bind at a ratio of at least one peptide per fibril.

FIG. 11 shows testing of co-localization of HD 1/HD 8 and Httex1. In these experiments, N2A cells (mouse neuroblastoma cell line) were transfected with Httex1 Q39 fused with RFP (middle column). Cells were fixed and stained for HD 1 or HD 8 with Alexa 488 (left column). Co-localization of HD1/HD8 with Httex1 aggregates is shown in the merged image (co-localization observed in cells is indicated by white arrows). The “CTRL” rows show the non-transfected cells, which were treated with 5 times more peptide to confirm the localization was not due to aggregations of peptides or off-target binding occurring in the cell.

FIG. 12 is a bar graph showing alanine scanning of HD1.

FIG. 13 is a bar graph showing alanine scanning of HD8.

FIG. 14 presents a bar graph and sequence alignments showing mutational scanning of HD1

FIG. 15 presents bar graphs and sequence alignments showing testing of mutations of the C-terminal constant region of HD1.

FIG. 16 presents bar graphs showing and sequence alignments testing of mutations of the C-terminal constant region of HD8.

FIG. 17 presents a bar graph and sequence alignments showing testing dimeric pairs of peptide agents using HD1 or HD8 peptides. The experiments were performed with 20 μL Streptavidin UltraLink Beads Slurry in 1 mL buffer, 4 μg protein (about 280 pmol as monomer).

FIG. 18 is a bar graph showing testing of dimeric pairs of peptide agents using HD1 or HD8 peptides.

FIG. 19 is a bar graph showing testing of the effect of urea on the dimeric HD8-SP10-HD1 peptide agent.

FIG. 20 is a bar graph showing testing of the effect of decreased amounts of fibril immobilized on beads using the dimeric HD8-SP10-HD1 peptide agent.

FIG. 21 is a bar graph showing a comparison of monomeric, dimeric, and trimeric peptide agents using lower fibril conditions. The experiments were performed with 10 μL Streptavidin UltraLink Beads Slurry in 1 mL buffer, 0.7 μg protein (about 50 pmol as monomer).

FIG. 22 is a bar graph showing a comparison of monomeric, dimeric, trimeric, and tetrameric peptide agents using lower fibril conditions. The experiments were performed with 10 μL Streptavidin UltraLink Beads Slurry in 1 mL buffer, 0.5 μg protein (about 35 pmol as monomer).

FIG. 23 is a bar graph showing that multimerization of QBP1 yielded higher affinity peptide agents. The experiments were performed with 20 μL Beads Slurry in 1 mL buffer, 2 μg protein (about 140 pmol as monomer).

FIG. 24 is a bar graph showing EPR based determination of aggregation inhibition by peptides. In this figure, seeded Httex1 aggregation at room temperature in the presence of the indicated amounts of peptides was quantified by EPR. Percent inhibition was obtained from the amplitude change of the aggregation reaction in the absence or presence of peptides (Httex1 concentration 15 μM).

FIG. 25 presents bar graphs showing testing of binding affinity in the presence of serum. In these experiments, serum was diluted 1:2, 1:5, or 1:10 with binding buffer (TBST) and HD1 (top panel) or HD8-SP10-HD1-SP10-HD8-SP10-HD1 (bottom panel) was tested for binding. HD1 binding was performed with 20 μL Beads Slurry in 1 mL buffer, 2 μg protein (about 140 pmol as monomer) while the HD8/HD1 tetramer binding was performed with 10 μL Beads Slurry in 1 mL buffer, 0.5 μg protein (about 35 pmol as monomer).

FIG. 26 presents fluorescence microscopy pictures showing co-expression of GFP-HD8-SP10-HD1 and Httex1Q72 in HEK293T cells lowered Httex1Q72 expression. In these experiments, HEK293T cells were co-transfected construct to express Httex1Q72 appended with red fluorescent protein (RFP), without (panels A1-A3 and C1-C3) or with GFP-HD8-SP10-HD1 (panels B1-B3 and D1-D3). After 24 h, cells were fixed and imaged for nuclei (DAPI, shown in panels A1, B1, C1 and D1) to visualize all cells, and imaged for RFP to visualize cells that express Httex1Q72-RFP (shown in panels A3, B3, C3 and D3), and imaged for GFP to visualize cells that express GFP-HD8-SP10-HD1 (shown in panels A2, B2, C2 and D2). Two different laser power settings were used to capture a wider range of fluorescent signal (1×, top panels and 10×, bottom panels). The expression of GFP-HD8-SP10-HD1 lowered the amount of Httex1Q72-RFP signal when compared to cells not expressing the peptide construct (compare panels A1-A3 to B1-B3 and compare panels C1-C3 to D1-D3).

FIG. 27 presents microscopy pictures showing that HD8-SP10-HD1-fluorescein bound Httex1Q72 aggregates in HEK293T cells. Left panels show fluorescent images (top left panel: nuclear stain to visualize all cells; middle left panel: fluorescein signal arising from the HD8-SP10-HD1-fluorescein peptide; bottom left panel: antibody against fluorescein to further amplify fluorescein signal). Right panels show phase contrast image of the same field. Arrows point to cytoplasmic aggregates that could be visualized with fluorescent signals or under phase contrast. Asterisks mark intranuclear aggregates that were labeled with HD8-SP10-HD1-fluorescein but not anti-fluorescein antibody.

FIG. 28 presents fluorescence microscopy pictures showing that HD8-Alexa488 labels aggregated Httex1 in retina sections from two different mouse model of Huntington disease. Left panels show nuclear stain that labels all cells. Right panels show signal from HD8-Alexa488. Top panels show a retinal section from a R6/1 mouse, and bottom panels show a retinal section from a zQ175KI mouse. Specific staining was observed in the ganglion cell layer of the retina in both mouse models. Little or no labeling was observed in other retinal layers.

FIG. 29 presents fluorescence microscopy pictures showing that HD8-SP10-HD1-fluorescein showed enhanced reactivity of Httex1 aggregates in all retinal layers of the R6/1 Huntington mouse model. Left panels show nuclear stain that labels all cells (A1 and B1), and right panels show signal from HD8-SP10-HD1-fluorescein (A2 and B2). Top panels are higher magnification that show the ganglion cell layer of the retina, and the bottom panels are lower magnification micrographs that show all retinal layers. Labeling of all retinal layers is seen when the HD peptides are dimerized. The phase contrast panel to the left of the bottom panels show the corresponding retinal layers: ONL: outer nuclear layer that contain rod and cone photoreceptor cells, INL: inner nuclear layer that contains cell bodies from horizontal cells, bipolar cells, amacrine cells and Müller cells, GCL: ganglion cell layer.

FIG. 30 presents fluorescence microscopy pictures showing HD8-SP10-HD1-fluorescein labels Httex1 aggregates in brain section from R6/1 mouse model of Huntington disease. Brain sections from age-matched littermate control and R6/1 mouse are shown in this figure. The control section showed nuclear stain that labels all cells, and the R6/1 brain showed cell nuclei in addition to signal from HD8-SP10-HD1-fluorescein (indicated by arrows).

### DETAILED DESCRIPTION

Presented herein are novel peptides and peptide agents and uses thereof, for example, for preventing or treating a subject having, or suspected of having HD. In certain embodiments, the peptide agents described herein are capable of selectively binding to unbundled fibrils of Huntingtin exon 1. Peptide agents that are capable of specifically recognizing Huntingtin fibrils are useful in several applications as described below.

In some embodiments, the peptide agents are useful for therapeutic purposes to block the aggregation of Huntingtin and to prevent the undesirable effects of such aggregation. In certain embodiments, peptides or peptide agents comprises a peptide sequence having 40%, 50%, 60%, 70%, 80%, 90% or more identity to a peptide disclosed herein. In certain embodiments, peptides or peptide agents comprise one or more of the 20 natural amino acids. In another embodiment, the peptides or peptide agents comprise unnatural amino acids, amino acid derivatives or analogues that include, but are not limited to, one or more of N-methyl amino acids, C-alpha amino acids, Beta amino acids, D-amino acids, or Peptide-nucleic acids. It is also recognized by one skilled in the art that other unnatural alpha amino acids could be substituted for amino acids in these peptide agents, for example substituting norleucine or norvaline for methionine. One skilled in the art can also recognize that truncations of these peptides (either from the N- or C-terminus) or extensions of these peptides (either from the N-or C-terminus) can also result in similar peptide function. In some embodiments, these truncations are desirable in order to minimize the size of peptide to increase therapeutic efficiency, cell permeability, to simply synthesis, decrease cost, etc. In other embodiments, extension of the peptides, including the use of a fusion protein (e.g., MBP, thioredoxin, ubiquitin, SUMO, NusA, GST, GFP, etc.) are also desirable, for example, to generate constructs that can be used to study Huntingtin function inside a cell.

In another embodiment these peptide agents can be used as diagnostics for Huntington's Disease. In some cases, it is useful to detect the presence of Huntingtin fibrils present in body fluids (including but not limited to blood, serum, tears, mucosal fluid, urine, intestinal fluid, cerebrospinal fluid, urine, sweat, semen, or vaginal fluids). In other cases, these peptide agents can be used to detect the presence of Huntingtin fibrils in the body using imaging techniques, for example using fluorescence, UV, visible, IR, near-IR, X-ray, magnetic resonance, positron emission tomography, cryo-electron microscopy, or others. In one embodiment, a peptide-conjugate could be used to deliver a small molecule or other useful molecule enabling imaging experiments. For example, a peptide could be conjugated to a fluorescent dye, a near-IR dye, an MRI contrast reagent, a nanoparticle, or a radiotracer.

In another embodiment the peptides disclosed herein are multimerized to increase the affinity of the peptide agents for Huntingtin fibrils. Higher affinity peptide agents are desirable in many different applications. For example, a higher affinity peptide agent can increase the potency of a therapeutic or reduce the amount of therapeutic that must be administered. Higher affinity peptide agents could also be useful in a diagnostic application to increase the sensitivity or specificity of a diagnostic test. It is thought that multimerizing the peptide agents increases the affinity since fibrils can present multiple binding sites so that the multiple peptide agents can bind simultaneously, though the validity of this mechanism does not affect the observed fact that multimerizing these peptide agents results in increase peptide affinity for fibrils. Multimerization can be achieved through the connection of one or more peptide agents for the fibrils. For example, dimeric peptide agents of the form: (peptide agent)-spacer-(peptide agent) or (peptide agent)-(peptide agent) have been developed in this application, where the (peptide agent) is a fibril binding molecule and the spacer enables two peptide agents to span the distance between two binding sites on a fibril. The spacer can thus be composed of amino acids or could be any other linker that provides sufficient separation enabling multivalent peptide agent binding. In some cases, the use of Gly as a spacer is desirable, as Glycine provides flexibility in a linker. In other cases, the use of hydrophilic amino acids is desirable to improve the water solubility of the overall molecule. In other cases, the linker can be composed of amino acids that might increase the binding affinity of the overall molecule. In other embodiments, the linker is composed of a Polyethylene glycol spacer (PEG), which is a commonly used spacer in the art.

These peptides can also find use in a surrogate peptide agent screen in order to discover small molecules that can bind Huntingtin fibrils. For example, a fluorescently-labeled peptide would first be bound to Huntingtin fibrils and small molecule libraries would be screened for molecules that displace the fluorescent peptides. These small molecules would then be lead compounds for further therapeutic development.

Subjects

The term “subject” refers to animals, typically mammalian animals. In some embodiments a subject is a mammal. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a subject is a primate. In some embodiments a subject is a human. A mammal can be any age or at any stage of development (e.g., an adult, teen, child, infant, or a mammal in utero). A mammal can be male or female. In certain embodiments a mammal can be an animal disease model, for example, animal models used for the study of HD.

In certain embodiments a subject has or is suspected of having HD. In certain embodiments a subject is at risk of developing HD. Subjects at risk of developing HD can be subjects in high risk groups who can be identified by a medical professional.

Peptide Agents

In certain embodiments, a peptide agent comprises one or more peptides disclosed herein. In some embodiments, peptide agents selectively bind to unbundled fibrils of the exon-1 fragment of Huntingtin (Httex1). In some embodiments, a peptide agents two or more peptides that are multimerized to generate even higher affinity peptide agents for Httex1 unbundled fibrils. The peptide agents disclosed here are potential lead compounds for therapeutics and diagnostics to treat Huntington's disease.

Presented herein, in certain embodiments, are methods of treating a subject having, or suspected of having a HD, which methods comprise administering to a subject in need thereof, a therapeutically effective amount of one or more peptide agents, or a therapeutically effective amount of a pharmaceutical composition comprising one or more peptide agents.

A peptide agent, in some embodiments, comprises a length of between 10 and 500 amino acids, 10 and 100 amino acids, between 10 and 50 amino acids, between 10 and 30 amino acids, or between 10 and 12 amino acids. In some embodiments, a length of a peptide agent is 8, 9, 10, 11, 12, 13, 14, or 15 amino acids long. In some embodiments, peptide agents can be provided in mixtures or compositions comprising two or more peptide agents. In some embodiments, where two or more peptide agents are present, the peptide agents can be arranged in a polypeptide in tandem where the two or more HD peptides are linked by a covalent bond (e.g., a peptide bond or other suitable linkage).

In certain embodiments, a peptide agent comprises or consists of a peptide having an amino acid sequence selected from MDWWPMWPSL (HD1; SEQ ID NO:1); MWMIQMPGYQ (HD2; SEQ ID NO:2); MRWSMSYSWA (HD3; SEQ ID NO:3); MFMMMWMSLT (HD4; SEQ ID NO:4); MFFVLSWTPL (HD5; SEQ ID NO:5); MQMWTMWEPW (HD6; SEQ ID NO:6); MDLWPMWESW (HD7; SEQ ID NO:7); MWQMMNGMSQ (HD8; SEQ ID NO:8); MX2X3X4X5X6X7X8X9X10 (HD1-Derivative; SEQ ID NO:9), where X2 is selected from D, H, L, N, T and Y, X3 is selected from F, R and W, X4 is selected from R and W, X5 is selected from H, P, S and T, X6 is selected from L, M, Q, S, T and V, X7 is selected from R and W, X8 is selected from A, E, H, P, R, S, and T, X9 is selected from D, P, Q, S, T and W, and X10 is selected from L, M, P, R and T; MX2X3WX5X6WX8X9X10 (HD6/7-Derivative; SEQ ID NO:10), wherein X2 is selected from A, D, E, I, L, M, N, P, Q, T and V, X3 is selected from F, L, M, S, W and Y, X5 is selected from A, D, E, G, N, P, Q and T, X6 is selected from F, L, M and W, X8 is selected from D, E, N, Q and P, X9 is selected from C, D, N, P, S and T, and X10 is selected from L and W; MWX3X4X5X6X7X8X9Q (HD2-Derivative; SEQ ID NO:11), wherein X3 is selected from M and Q, X4 is selected from I and M, X5 is selected from M and Q, X6 is selected from F and M, X7 is selected from I, M, N, P and Q, X8 is selected from F, G, M, S and W, and X9 is selected from E and Y; and MX2QX4MX6X7X8X9Q (HD8-Derivative; SEQ ID NO:12), wherein X2 is selected from F, W and Y, X4 is selected from L, M, W and Y, X6 is selected from G, H, N, S and Y, X7 is selected from G and N, X8 is selected from I and M, and X9 is selected from G, S and Y. In certain embodiments, a peptide agent comprises or consists of a peptide having an amino acid sequence selected from MAWWPMWPSL (HD1-D2A; SEQ ID NO:13), MDAWPMWPSL (HD1-W3A; SEQ ID NO:14), MDWWAMWPSL (HD1-P5A; SEQ ID NO:15), MDWWPAWPSL (HD1-M6A; SEQ ID NO:16), MDWWPMWASL (HD1-P8A; SEQ ID NO:17), MDWWPMWPAL (HD1-S9A; SEQ ID NO:18), and MDWWPMWPSA (HD1-L10A; SEQ ID NO:19). In certain embodiments, a peptide agent comprises or consists of a peptide having an amino acid sequence selected from MDWWPLWPSL (HD1-M6L; SEQ ID NO:20), MDWWPTWPSL (HD1-M6T; SEQ ID NO:21), MDWWPVWPSL (HD1-M6V; SEQ ID NO:22), MMDWWPMWPSL (HD1-MM; SEQ ID NO:23), MLDWWPMWPSL (HD1-ML; SEQ ID NO:24), MMDWWPLWPSL (HD1-MM6L; SEQ ID NO:25), and MLDWWPLWPSL (HD1-ML6L; SEQ ID NO:26). In certain embodiments, a peptide agent comprises or consists of a peptide having an amino acid sequence selected from MAQMMNGMSQ (HD8-W2A; SEQ ID NO:27), MWAMMNGMSQ (HD8-Q3A; SEQ ID NO:28), MWQAMNGMSQ (HD8-M4A; SEQ ID NO:29), MWQMANGMSQ (HD8-M5A; SEQ ID NO:30), MWQMMAGMSQ (HD8-N6A; SEQ ID NO:31), MWQMMNAMSQ (HD8-G7A; SEQ ID NO:32), MWQMMNGASQ (HD8-M8A; SEQ ID NO:33), and MWQMMNGMAQ (HD8-S9A; SEQ ID NO:34). In certain embodiments, a peptide agent comprises or consists of a peptide having an amino acid sequence selected from MWQMMNGMSQ GSGTSGSS (SEQ ID NO:39); MWQMMNGMSQA (HD8A; SEQ ID NO:40); MWQMMNGMSQG (HD8G; SEQ ID NO:41); MWQMMNGMSQGS (HD8-GS; SEQ ID NO:42); MWQMMNGMSQ ASGTSGSS (HD8-G11A; SEQ ID NO:43); MWQMMNGMSQ GAGTSGSS (HD8-G13A; SEQ ID NO:44); MMWQMMNGMSQ GSGTSGSS (HD8MM; SEQ ID NO:45) and MAWQMMNGMSQ GSGTSGSS (HD8MA; SEQ ID NO:46).

In certain embodiments, a peptide agent comprises or consists of 2 to 50 peptides having an amino acid sequence selected from SEQ ID NOs:1-34 and 39-46. In certain embodiments, a peptide agent comprises or consists of two, three or four peptides having an amino acid sequence selected from SEQ ID NOs:1-34 and 39-46.

In some embodiments a peptide agent comprises one or more linkers. In some embodiments, a linker covalently connects two peptides. In some embodiments, a linker comprises or consists of an amino acid selected from A, G and S, or an amino acid sequence selected from GS, GT, GSG, GSGT (SEQ ID NO:47), GSGTS (SEQ ID NO:48), GSGTSGSS (SEQ ID NO:35), ASGTSGSS (SEQ ID NO:36), GAGTSGSS (SEQ ID NO:37), GSATSGSS (SEQ ID NO:38) and GSGTSGSSGS (SEQ ID NO:50).

In some embodiments a peptide agent comprises a label. As used herein, the terms “label” or “labeled” refers to incorporation of a detectable marker, e.g., by incorporation of a labeled amino acid or attachment to a polypeptide of biotin moieties that can be detected by labeled avidin (e.g., streptavidin containing a fluorescent marker or enzymatic activity that can be detected by optical or colorimetric methods). In certain embodiments, a label or marker can be attached to a peptide agent to generate a therapeutic or diagnostic agent. A peptide agent can be attached covalently or non-covalently to any suitable label or marker. Various methods of labeling polypeptides and glycoproteins are known to those skilled in the art and can be used. Non-limiting examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹²⁵I, ¹³¹I), fluorescent labels, enzymatic labels (e.g., horseradish peroxidase, β-galactosidase, luciferase, alkaline phosphatase), chemiluminescent labels, a metallic label, a chromophore, an electro-chemiluminescent label, a phosphorescent label, a quencher (e.g., a fluorophore quencher), a fluorescence resonance energy transfer (FRET) pair (e.g., donor and acceptor), a dye, an enzyme substrate, a small molecule, a mass tag, quantum dots, nanoparticles, biotinyl groups, predetermined polypeptide epitopes recognized by a secondary reporter (e.g., leucine zipper pair sequences, binding sites for secondary antibodies, metal binding domains, epitope tags), the like or combinations thereof.

In some embodiments a peptide agent comprises a suitable therapeutic agent. A peptide agent can be attached covalently or non-covalently to any suitable therapeutic agent. Non-limiting examples of a therapeutic agent include a medication, toxin, radioisotope, peptide agent, receptor, cytokine, antibody, anti-neoplastic agent, inhibitor (e.g., a receptor antagonist, an enzyme inhibitor), a cytokine or an agent disclosed in U.S. Pat. No. 6,660,843, which is incorporated herein by reference, the like or combinations thereof. Non-limiting examples of anti-neoplastic agents include an auristatin (e.g., monomethyl auristatin E (MMAE), monomethyl auristatin F (MMAF), and the like), a dolastatin, a maytansine, a tubulysin, an irinotecan or derivative or metabolite thereof (e.g., SN38), a calicheamicin, a pyrrolobenzodiazepine (PBD), a duocarmycin, a doxorubicin, a pseudomonas exotoxin A (e.g., PE38), derivatives thereof, the like or combinations thereof. Accordingly, in certain embodiments, a peptide agent disclosed herein comprises an anti-neoplastic agent.

In some embodiments a peptide agent comprises a suitable carrier. A peptide agent can be attached covalently or non-covalently to a suitable carrier. Suitable carriers include agents or molecules that alter or extend the in vivo half-life of a peptide agent, non-limiting examples of which include polyethylene glycol, glycogen (e.g., by glycosylation of a peptide agent), a dextran, a carrier or vehicle described in U.S. Pat. No. 6,660,843, the like or combinations thereof.

In some embodiments a label, therapeutic agent or carrier is bound to a peptide agent by use of a suitable linker. Non-limiting examples of a suitable linker include silanes, thiols, phosphonic acid, polyethylene glycol (PEG), amino acids and peptides, polymers thereof, derivatives thereof, the like and combinations thereof. Methods of attaching two or more molecules using a linker are known to those skilled in the art and are sometimes referred to as “crosslinking.”

In some embodiments a label, therapeutic agent, carrier or linker is attached to a suitable thiol group of a peptide agent (e.g., a thiol group of a cysteine residue). In some embodiments, a thiol group is added to a peptide agent (e.g., by addition of a cysteine residue). Other non-limiting examples of attaching a label, therapeutic agent, carrier and/or linker to a peptide agent include reacting an amine with an N-hydroxysuccinimide (NHS) ester, an imidoester, a pentafluorophenyl (PFP) ester, a hydroxymethyl phosphine, an oxirane or any other carbonyl compound; reacting a carboxyl with a carbodiimide; reacting a sulfhydryl with a maleimide, a haloacetyl, a pyridyldisulfide, and/or a vinyl sulfone; reacting an aldehyde with a hydrazine; reacting any non-selective group with diazirine and/or aryl azide; reacting a hydroxyl with isocyanate; reacting a hydroxylamine with a carbonyl compound; the like and combinations thereof.

In certain embodiments a peptide agent is modified to include certain amino acid additions, substitutions, or deletions designed or intended, for example, to reduce susceptibility of a peptide agent to proteolysis, reduce susceptibility of a peptide agent to oxidation, increase serum half-life and/or confer or modify other physicochemical, pharmacokinetic or functional properties of a peptide agent. In certain embodiments a peptide agent is modified to include certain amino acid additions, substitutions, or deletions designed or intended to increase efficacy (e.g., the anti-metastatic properties) of a peptide agent. Accordingly, a peptide agent may comprise or consists of an amino acid sequence that is at least 70%, at least 75%, at least 80%, at least 85%, at least 90% or at least 95% identical (e.g., percent sequence identity) to a peptide agent of any one of SEQ ID NOs: 1 to 16. In some embodiments, a peptide agent shares at least 90% sequence identity to the amino acid sequence of a peptide agent described herein (e.g., any of SEQ ID Nos: 1 to 16).

The term “percent identical” or “percent identity” refers to sequence identity between two amino acid sequences. Identity can be determined by comparing a position in each sequence which may be aligned for purposes of comparison. When an equivalent position in the compared sequences is occupied by the same amino acid, then the molecules are identical at that position. When the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used to determine percent identity, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

In some embodiments, a peptide agent comprises one or more suitable modifications or modified amino acids non-limiting examples of which include D-amino acids, amino acids modified by acetylation, acylation, phosphorylation, glycosylation, myristoylation, amidation, hydroxylation (e.g., aspartic acid/asparagine hydroxylation), phosphopantethane attachment, methylation, methylthiolation, prenylation, addition of an intein, ADP-ribosylation, bromination, citrullination, deamination, dihdroxylation, formylation, geranyl-geranylation, glycation or palmitoylation.

Pharmaceutical Compositions, Administration and Dosing

In certain embodiments, a pharmaceutical composition comprises one or more peptide agents. In certain embodiments, a pharmaceutical composition comprises at least 2, at least 3 or at least 4 peptide agents. In certain embodiments, a pharmaceutical composition comprises 1 to 20, 1 to 10, or 1 to 5 peptide agents. In certain embodiments, a pharmaceutical composition comprises 1, 2, 3, 4, 5, 6, 7, 8 or more peptide agents.

In some embodiments, a pharmaceutical composition is delivered to a subject or HD cell via one or more delivery systems depending on the indication, disease state, severity, clinical utility and other relevant parameters that may impact the desired efficacy of a treatment using one or more peptide agents described herein.

The exact formulation and route of administration of a peptide agent (e.g., one or more peptide agents) or a composition for use according to the methods of the invention described herein can be chosen by the individual physician in view of a patient's condition. See e.g., Fingl et al. 1975, in “The Pharmacological Basis of Therapeutics,” Ch. 1 p. 1; which is incorporated herein by reference in its entirety. Any suitable route of administration can be used for administration of a composition (e.g., a pharmaceutical composition) or a peptide agent described herein. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranially, intraarticular, into a joint space, intracardiac (into the heart), intracavernous injection, intralesional (into a skin lesion), intraosseous infusion (into the bone marrow), intrathecal (into the spinal canal), intrauterine, intravaginal, intratumoral, intravesical infusion, intravitreal), the like or combinations thereof.

In some embodiments one or more peptide agents or a composition described herein is provided to a subject. A composition that is provided to a subject is sometimes provided to a subject for self-administration or for administration to a subject by another (e.g., a non-medical professional). For example a composition described herein can be provided with an instruction written by a medical practitioner that authorizes a patient to be provided a composition or treatment described herein (e.g., a prescription). In another example, a composition can be provided to a subject wherein the subject self-administers a composition orally, intravenously, topically or by way of an inhaler, for example.

One or more peptide agents and compositions (e.g., compositions comprising a one or more peptide agents) can be formulated to be compatible with a particular route of administration or use. Compositions for parenteral, intradermal, or subcutaneous administration can include a sterile diluent, such as water, saline solution, fixed oils, polyethylene glycols, glycerin, propylene glycol or other synthetic solvents. A pharmaceutical composition may contain one or more preservatives to prevent microorganism growth (e.g., antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose). Antibacterial and antifungal agents include, for example, parabens, chlorobutanol, phenol, ascorbic acid and thimerosal.

Compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). The carrier can be a solvent or dispersion medium containing, for example, water, ethanol, polyol (e.g., glycerol, propylene glycol, and polyethylene glycol), and suitable mixtures thereof. Fluidity can be maintained, for example, by the use of a coating such as lecithin, or by the use of surfactants. In some embodiments, a pharmaceutical composition includes an agent that delays absorption, for example, aluminum monostearate and gelatin which can prolong absorption of injectable compositions. In some embodiments, a pharmaceutical composition comprises polysorbate 20 or polysorbate 80, for example, up to 1%. Other non-limiting additives include histidine HC1, and α,α-trehalose dehydrate.

In some embodiments, one can administer compositions for use according to the methods of the invention in a local rather than systemic manner, for example, via direct application to the skin, mucous membrane or region of interest for treating, including using a depot or sustained release formulation.

In some embodiments, active ingredients (e.g., one or more peptide agents) can be administered alone or formulated as a composition (e.g., a pharmaceutical composition). In other embodiments, a one or more peptide agents can be administered in combination with one or more additional materials (e.g., one or more chemotherapeutic agents or cytokines), for example, as two separate compositions or as a single composition where the additional material(s) is (are) mixed or formulated together with a one or more peptide agents. For example, without being limited thereto, a one or more peptide agents can be formulated with additional excipients, or additional active ingredients.

The pharmaceutical compositions can be manufactured by any suitable manner, including, e.g., by means of conventional mixing, dissolving, granulating, dragee-making, levigating, emulsifying, encapsulating, entrapping or tableting processes.

Pharmaceutical compositions comprising one or more peptide agents described herein for use in accordance with the invention can be formulated in any suitable manner using one or more pharmaceutically acceptable carriers, solvents, salts, excipients, additives, preservatives, and/or auxiliaries. Proper formulation can depend upon the route of administration chosen. In particular, a pharmaceutical compositions can comprise any suitable formulation, ingredient, excipient, the like or combinations thereof as listed in “Remington's Pharmaceutical Sciences,” Mack Publishing Co., Easton, PA, 18th edition, 1990. can be used with a composition described herein. The various materials listed herein, alone or in combination, can be incorporated into or used with the materials described in Remington's. Any suitable techniques, carriers, and excipients can be used, including those understood in the art; e.g., in Remington's Pharmaceutical Sciences, above.

In some embodiments, a pharmaceutical composition comprising one or more peptide agents described herein can be formulated, for example, as a topical formulation. The topical formulation may include, for example, a formulation such as a gel formulation, a cream formulation, a lotion formulation, a paste formulation, an ointment formulation, an oil formulation, and a foam formulation. The composition further may include, for example, an absorption emollient.

In some embodiments, at least part of the affected area of a mammal (e.g., a melanoma or surface-exposed HD) is contacted with a composition on a daily basis, on an as-needed basis, or on a regular interval such as twice daily, three times daily, every other day, etc. A composition comprising one or more peptide agents can be administered for a period of time ranging from a single as needed administration to administration for 1 day to multiple years, or any value there between, (e.g., 1-90 days, 1-60 days, 1-30 days, etc.). The dosages described herein can be daily dosages or the dosage of an individual administration, for example, even if multiple administrations occur (e.g., 2 sprays into a nostril).

Some embodiments relate to methods of treating or preventing HD through administration of compositions comprising one or more peptide agents described herein to the upper respiratory track/bronchi in a mammal in need thereof, for example, by contacting at least part of the upper respiratory tract/bronchi of a mammal with a therapeutically effective amount of a composition as disclosed above or elsewhere herein. The composition can be, for example, formulated as an aerosol formulation, including formulated for use in a nebulizer or an inhaler. The compositions may include, for example, one or more of dichlorodifluoromethane, trichlorofluoromethane, dichlorotetrafluoroethane, carbon dioxide, and the like. The pharmaceutical compositions can be formulated for use in a nebulizer or an inhaler, for example.

A pharmaceutical composition may comprise one or more suitable carriers. In some embodiments, a carrier includes one or more chemical compounds that facilitate the incorporation of an active ingredient (e.g., one or more peptide agents) into cells or tissues. For example dimethyl sulfoxide (DMSO) is a commonly utilized carrier as it facilitates the uptake of many compounds and peptides into the cells or tissues of an organism. In some embodiments, a pharmaceutical carrier for a composition described herein can be selected from castor oil, ethylene glycol, monobutyl ether, diethylene glycol monoethyl ether, corn oil, dimethyl sulfoxide, ethylene glycol, isopropanol, soybean oil, glycerin, zinc oxide, titanium dioxide, glycerin, butylene glycol, cetyl alcohol, and sodium hyaluronate.

In certain embodiments, a pharmaceutical composition comprises hydrophobic excipients, additives, or other hydrophobic components. A pharmaceutical carrier for certain hydrophobic peptides can be a co-solvent system comprising benzyl alcohol, a nonpolar surfactant, a water-miscible organic polymer, and an aqueous phase. A common co-solvent system contemplated for use herein is the VPD co-solvent system, which is a solution of 3% w/v benzyl alcohol, 8% w/v of the nonpolar surfactant POLYSORBATE 80™, and 65% w/v polyethylene glycol 300, made up to volume in absolute ethanol. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of POLYSORBATE 80™; the fraction size of polyethylene glycol can be varied; other biocompatible polymers can replace polyethylene glycol, e.g., polyvinyl pyrrolidone; and other sugars or polysaccharides can substitute for dextrose.

Alternatively or additionally, other carriers can be employed, if required. Liposomes and emulsions are well known examples of delivery vehicles or carriers for hydrophobic drugs and drug compositions. Additionally, the one or more peptide agents described herein can be delivered using a sustained-release system, such as semipermeable matrices of solid hydrophobic polymers containing the therapeutic agent. The pharmaceutical compositions described herein can be administered to a patient per se, or in pharmaceutical compositions where they are mixed with other active ingredients, as in combination therapy, or suitable carriers or excipient(s). The compounds and compositions can be formulated with salts or excipients, such as for example, sodium or meglumine. Techniques for formulation and administration of the one or more peptide agents of the instant application can be found in “Remington's Pharmaceutical Sciences,” Mack Publishing Co., Easton, PA, 18th edition, 1990.

Furthermore, the compounds and compositions used herein can be stable over an extended period of time, for example on the order of months or years. Compositions described herein, in some embodiments, may comprise a preservative. The preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). The preservative can comprise an alkyl-mercury salt of thiosalicylic acid, such as thiomersal, phenylmercuric nitrate, phenylmercuric acetate or phenylmercuric borate. The preservative can comprise parabens, such as methylparaben or propylparaben. The preservative can comprise an alcohol, such as chlorobutanol, benzyl alcohol or phenyl ethyl alcohol. The preservative can comprise a biguanide derivative, such as chlorohexidine or polyhexamethylene biguanide. The preservative can comprise sodium perborate, imidazolidinyl urea, and/or sorbic acid. The preservative can comprise stabilized oxychloro complexes, such as known and commercially available under the trade name PURITE®). The preservative can comprise polyglycol-polyamine condensation resins, such as known and commercially available under the trade name POLYQUART® from Henkel KGaA. The preservative can comprise stabilized hydrogen peroxide generated from a source of hydrogen peroxide for providing an effective trace amount of resultant hydrogen peroxide, such as sodium perborate tetrahydrate. The preservative can be benzalkonium chloride.

The preservative can enable a composition to be used on multiple occasions. The preservative can reduce the effects of one or more of acid exposure, base exposure, air exposure, heat, and light on the active ingredient. The compounds and pharmaceutical compositions described herein can include any suitable buffers, such as for example, sodium citrate buffer and/or sequestering agents, such as edetate disodium sequestering agent. Ingredients, such as meglumine, may be added to adjust the pH of a composition or compound described herein. Compounds and compositions described herein may comprise sodium and/or iodine, such as organically bound iodine. Compositions and compounds used herein may be provided in a container in which the air is replaced by another substance, such as nitrogen.

Certain embodiments provide pharmaceutical compositions comprising one or more peptide agents in an amount effective to achieve its intended purpose (e.g., a therapeutically effective amount). A “therapeutically effective amount” means an amount to prevent, treat, suppress, inhibit, reduce the severity of, delay the onset of, suppress or inhibit the growth or viability of a HD, metastasis of a HD or one or more symptoms associate with a HD. A symptom can be a symptom already occurring or expected to occur. Determination of a therapeutically effective amount is well within the capability of those skilled in the art (e.g., a medical practitioner), especially in light of the detailed disclosure provided herein.

In some embodiments, a therapeutically effective amount is an amount needed for a significant quantity of a pharmaceutical composition (or peptide agent therein) to contact a desired region or tissue where prevention or treatment of a HD is desired.

The resulting effect of a treatment herein, in certain embodiments, is to provide a delay in the onset of HD, to inhibit, suppress or eliminate one or more symptoms of HD, to reduce the frequency or severity of symptoms associated with a HD, to ameliorate symptoms associated with a HD, to improve patient comfort or function, to decrease, reduce or inhibit the re-occurrence of a given HD in a subject, to decrease the size of a tumor or HD, to decrease the amount of HD cells in a subject, to decrease the severity of a condition associated with a HD, or to eliminate a HD. The overall beneficial effect of a treatment described herein can be determined by comparing the condition or disease state of a subject who received a treatment described herein to one or more individuals who have not received treatment, or to the same patient prior to treatment, or after cessation of, treatment. A treatment may be complete (no detectable symptoms or HD) or partial, such that fewer symptoms or amounts of a HD are observed than would likely occur absent treatment.

Compositions described herein can be administered at a suitable dose, e.g., at a suitable volume and concentration depending on the route of administration. Within certain embodiments of the invention, dosages of an administered composition, one or more peptide agents or peptide can be from a concentration, for example, of 0.1 ng/kg to 100 mg/kg (e.g., amount of active ingredient/body weight of a subject), 0.1 ng/kg to 1 mg/kg, 0.1 ng/kg to 100 μg/kg, 0.001 mg/kg to 100 mg/kg, 0.001 mg/kg to 10 mg/kg, 0.001 mg/kg to 1 mg/kg, about 0.01 mg/kg to 100 mg/kg, or about 0.01 mg/kg to about 50 mg/kg. In certain embodiments a composition or one or more peptide agents described herein can be administered at a concentration of at least 0.01 mg/kg, at least 0.1 mg/kg, at least 1 mg/kg, at least 10 mg/kg, or at least 50 mg/kg. An active ingredient refers to a peptide agent or a mixture of two or more peptide agents. The concentrations recited above can refer to the concentration of a single peptide agent, the collective concentration of a mixture of peptide agents, or the concentration of each of two or more peptide agents (e.g., peptides in a pharmaceutical composition or mixture). In certain embodiments a composition or one or more peptide agents is administered to a concentration in a range of 0.1 mg /kg to 10 mg/kg body weight of a subject. Volumes suitable for intravenous administration are well known. For example, 0.1 ml-100 ml of a composition, one or more peptide agents or peptide can be safely administered intravenously to an adult human subject.

Kits

In some embodiments the compositions, formulations, combination products and materials described herein can be included as part of kits, which kits can include one or more of the compositions, or peptide agents described herein, formulations of the same, chemotherapeutic agents for combination treatments and products and other materials described herein. In some embodiments the kit comprises one or more a peptide agents, or a pharmaceutical composition comprising the same. In some embodiments a kit comprises one or more peptide agents as described herein and a chemotherapeutic agent. In some embodiments the products, compositions, kits, formulations, etc. can come in an amount, package, product format with enough medication (e.g., one or more peptide agents) to treat a patient for 1 day to 1 year, 1 day to 180 days, 1 day to 120 days, 1 day to 90 days, 1 day to 60 days, 1 day to 30 days, or any day or number of days there between.

In some embodiments, a kit comprises suitable packaging materials. A kit optionally includes a label or packaging insert including a description of the components or instructions for use in vitro, in vivo, or ex vivo, of the components therein. Exemplary instructions include instructions for a method, treatment protocol or therapeutic regimen described herein. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards. Labels or inserts can include identifying information of one or more components therein, dose amounts, clinical pharmacology of the active ingredient(s) including mechanism of action, pharmacokinetics (PK) and pharmacodynamics (PD). Labels or inserts can include information identifying manufacturer information, lot numbers, manufacturer location and date.

Labels or inserts can include information on a condition, HD, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Components of the kit can be enclosed within an individual container and all of the various containers can be within a single package. Invention kits can be designed for cold storage.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed.

### EXAMPLES

### Example 1. Peptides Isolated Using mRNA Display

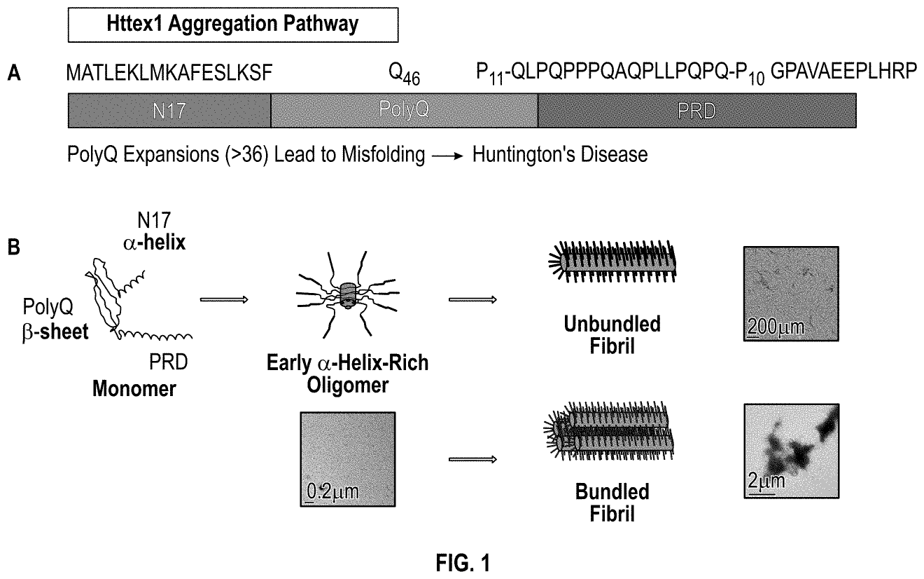

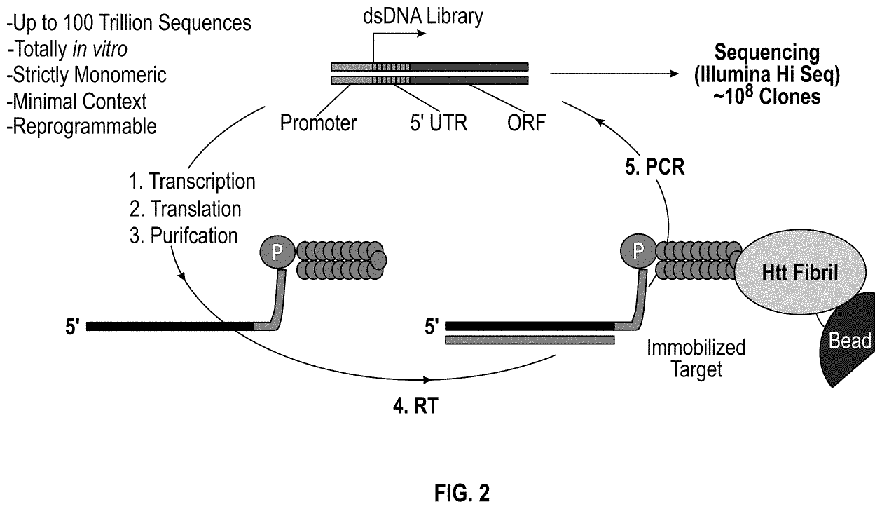

Messenger RNA (mRNA) display (described in (Roberts, et al. (1997) Proc Natl Acad Sci USA 94: 12297-302, Takahashi, et al. (2003) Trends Biochem Sci 28: 159-65); FIG. 2) was used to engineer peptide agents that bind to unbundled fibrils of Huntingtin exon 1. To do this, unbundled Huntingtin fibrils were generated by mixing biotinylated Huntingtin protein (exon 1; FIG. 1) with non-biotinylated exon 1 Huntingtin protein at a ratio of 1:20 (˜5% biotinylated Huntingtin). These fibrils were then immobilized on Neutravidin agarose (Thermo Fisher) or Streptavidin ultralink beads (Thermo Fisher) via the attached biotin. A MX₉ (where X is any of the 20 natural amino acids) mRNA display library was generated using published procedures (Takahashi, et al. (2009) Methods Mol Biol 535: 293-314) and incubated with immobilized fibrils. Non-binding sequences were washed away and the remaining fibril binding peptides were amplified via RT-PCR. This process was repeated seven times.

The binding of the peptide library was determined using a radioactive binding assay. In this assay, peptides were radiolabeled by translating the mRNA coding for these peptides in the presence of ³⁵S-methionine using rabbit reticulocyte lysate (Jackson, et al. (1983) Methods Enzymol 96: 50-74). These peptides were translated in an mRNA display format and were thus linked to their encoding mRNA, as described above. After purification by dT chromatography, the radiolabeled peptides were incubated with Huntingtin fibrils immobilized on Neutravidin Agarose or Streptavidin Ultralink beads. The beads were then filtered through a Spin X column (Corning) to separate the supernatant and beads, then washed to remove unbound sequences. The supernatant, washes, and beads were all counted in a scintillation counter to determine the counts in each sample. The percent bound was determined by dividing the counts on the beads with the total counts (beads+washes+supernatant). As a control, the same radiolabeled peptides were also incubated with beads containing no fibrils, in order to demonstrate that binding was specific to fibrils and not to the immobilization matrix.

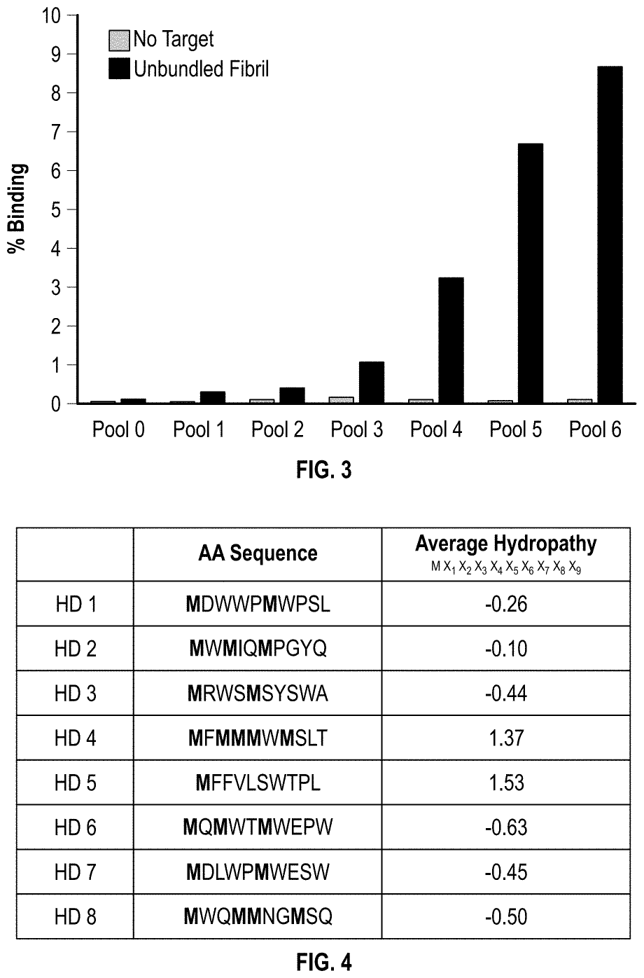

Pools 0 through 7 were tested using this radioactive binding assay (FIG. 3). In early rounds (Rds 1 and 2), only a little binding to fibrils above background was observed. In later rounds (Rds 3 through 7) significant binding was observed above the no target control, indicating that the peptides found in the library bind to unbundled Huntingtin fibril specifically.

### Example 2. Several Peptide Families Bind Htt-Fibrils Specifically

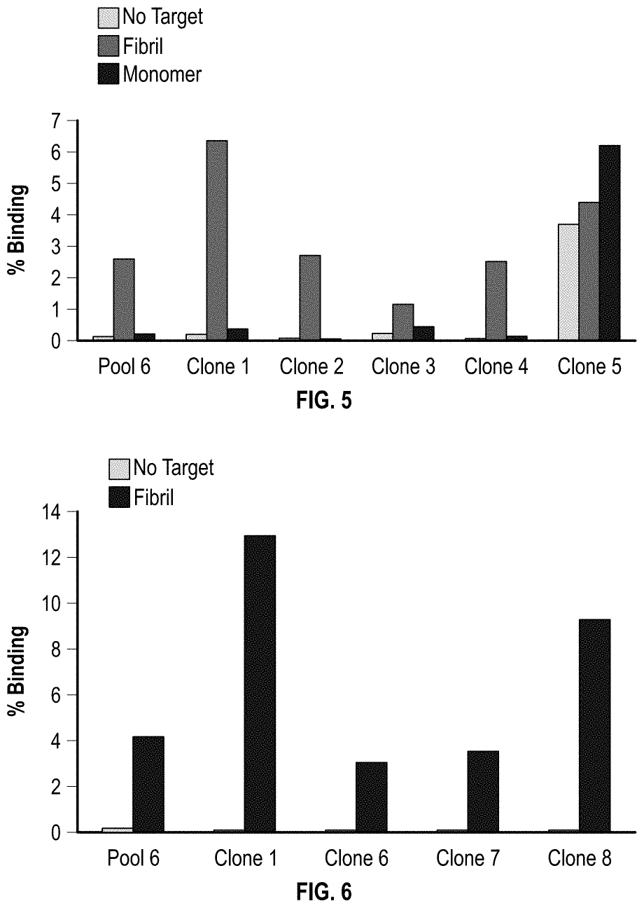

DNA sequencing of the Round 6 and Round 8 pool resulted in identification of thousands of putative Htt-fibril binding peptides. Eight clones (named HD1 through HD8; FIG. 4) were tested for binding to Htt-fibrils using the radioactive binding assay described above. FIG. 5 shows the binding of HD1 through HD5 to Htt-fibrils. HD1, HD2, HD3, and HD4 all demonstrated binding to Htt-fibrils with little to no binding to beads without target. HD5 showed high non-specific binding to beads without target and was not studied further. HD 6, HD7, and HD8 were also tested for binding and showed good binding to Htt-fibrils with little to no binding to beads without target (FIG. 6).

HD1 through HD4 were also tested for binding to monomeric Huntingtin (FIG. 5). HD1 through HD4 did not show appreciable binding to monomeric Huntingtin, demonstrating that the binding of these peptides was highly specific to the fibril form of the protein.

Further analysis of the Round 6 and Round 8 pools resulting from Illumina Sequencing using the program Hammock (Krejci, et al. (2016) Bioinformatics 32: 9-16) allowed the identification of homologous sequences to HD1 through HD8. These related sequences likely bind Htt-fibrils by nature of their presence in the DNA pools, as previous results showed that most, if not all, of the final clones in the library possess target binding capabilities (Cetin, et al. (2017) J Mol Biol 429: 562-573, Jalali-Yazdi, et al. (2016) Angew Chem Int Ed Engl 55: 4007-10). These additional sequences were of the following compositions:

### HD 1 Sequence Derivatives:

Position 1: M
Position 2: D, H, L, N, T, Y
Position 3: F, R, W
Position 4: R or W
Position 5: H, P, S, T
Position 6: L, M, Q, S, T, V
Position 7: R or W
Position 8: A, E, H, P, R, S, T
Position 9: D, P, Q, S, T, W
Position 10: L, M, P, R, T

### HD 6, HD 7 Sequence Derivatives:

Position 1: M
Position 2: A, D, E, I, L, M, N, P, Q, T, V
Position 3: F, L, M, S, W, Y
Position 4: W
Position 5: A, D, E, G, N, P, Q, T
Position 6: F, L, M, W
Position 7: W
Position 8: D, E, N, Q, P
Position 9: C, D, N, P, S, T
Position 10: L, W

### HD2 Sequence Derivatives:

Position 1: M
Position 2: W
Position 3: M, Q
Position 4: I, M
Position 5: M, Q
Position 6: F, M
Position 7: I, M, N, P, Q
Position 8: F, G, M, S, W
Position 9: E, Y
Position 10: Q

### HD8 Sequence Derivatives:

Position 1: M
Position 2: F, W, Y
Position 3: Q
Position 4: L, M, W, Y
Position 5: M
Position 6: G, H, N, S, Y
Position 7: G, N
Position 8: I, M
Position 9: G, S, Y
Position 10: Q

### Example 3. Characterization of HD1 and HD8

Two of the peptides that were identified to have the highest binding in the radioactive binding assay described above were selected for further characterization.

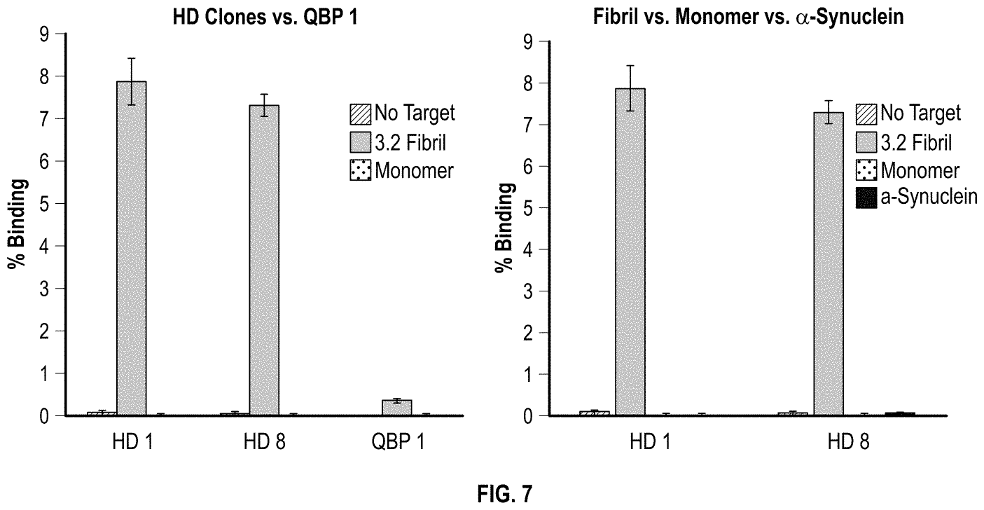

HD1 and HD8 were first tested for binding as mRNA-peptide fusions as described in Example 1. FIG. 7 shows that both HD1 and HD8 bind to Htt-fibrils with little to no binding to beads without target. HD1 and HD8 were compared to a previously reported peptide called QBP1 that was identified by phage display (Nagai, et al. (2000) J Biol Chem 275: 10437-42) to bind to aggregated polyglutamine proteins, such as Huntingtin fibrils. QBP1 showed binding above background to unbundled Htt-fibrils, but showed >10-fold less binding as compared with HD1 and HD8.

HD1 and HD8 were also tested for binding specificity. Radiolabeled HD1 and HD8 were incubated with monomeric Huntingtin, unbundled Htt-fibrils, or α-Synuclein fibrils (α-Synuclein is another protein known to aggregate and form fibrils). Both HD1 and HD8 show binding to Htt-fibrils and little to no binding to beads without target, monomeric Huntingtin, and α-Synuclein fibrils (FIG. 7), demonstrating that the interaction of these peptides is highly specific for Htt-fibrils.

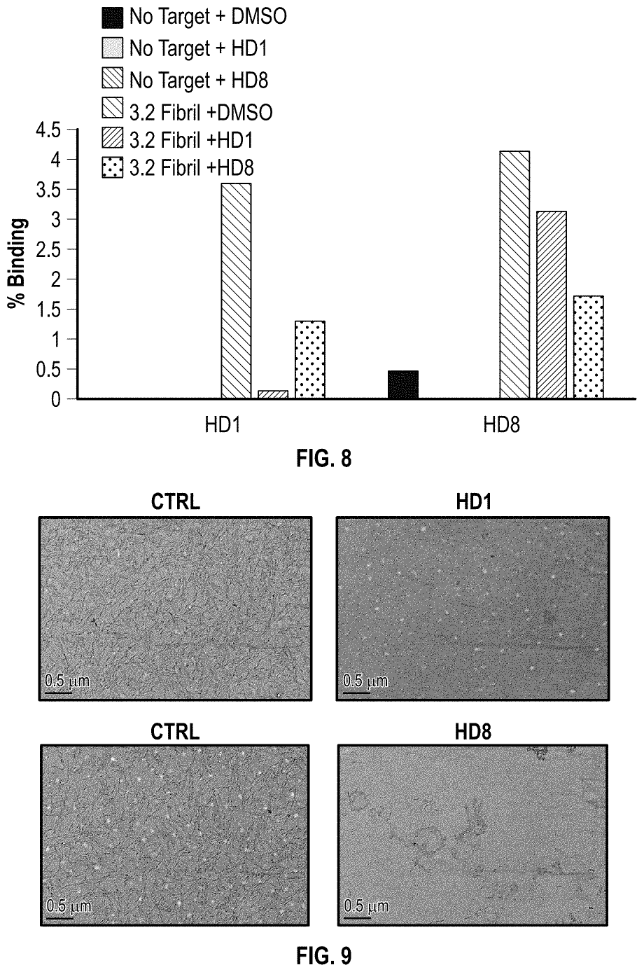

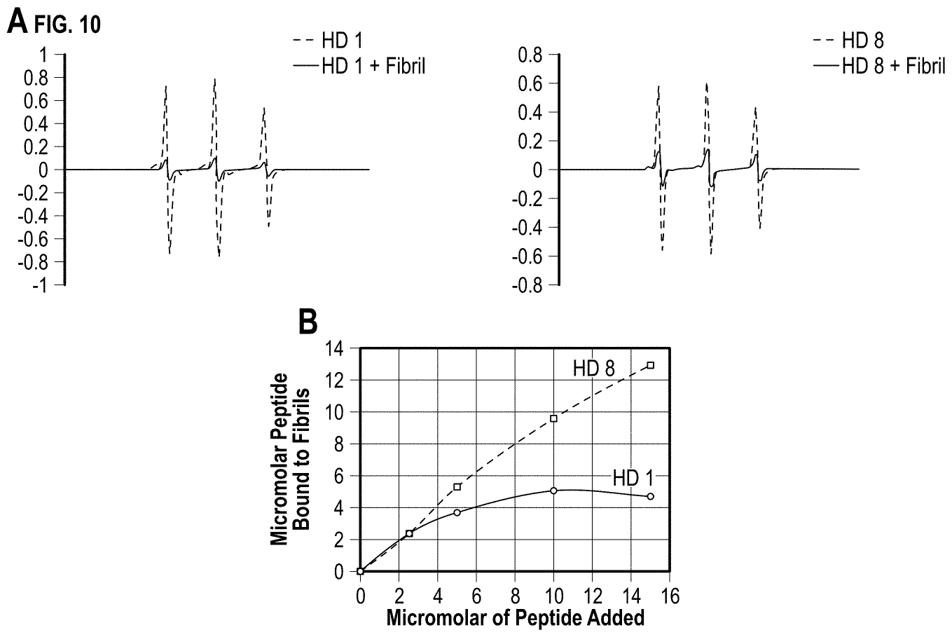

HD1 and HD8 were chemically synthesized and purified by reverse-phase HPLC and resuspended in DMSO. In order to verify that these synthetic peptides were functional for binding, we designed a competition experiment. We radiolabeled HD1 and HD8 above then tested to see if synthetic HD1 or HD8 would compete and reduce binding of the radiolabeled peptides. In FIG. 8, radiolabeled HD1 showed no binding to beads without target (No target+DMSO), to beads without target plus synthetic HD1 (No target+HD1) nor to beads without target plus synthetic HD8 (No target+HD8). Binding to Htt-fibrils (3.2 Fibril+DMSO) was reduced by the addition of synthetic HD1 (3.2 Fibril+HD1) or by addition of synthetic HD8 (3.2 Fibril+HD8). Similarly, radiolabeled HD8 showed low binding to beads without target (No target+DMSO) and little binding to beads with no target plus synthetic HD1 (No target+HD1) or to beads without target plus synthetic HD8 (No target+HD8). Radiolabeled HD8 binding to Htt-fibrils (3.2 Fibril+DMSO) was similarly reduced by the addition of synthetic HD1 (3.2 Fibril+HD1) or by addition of synthetic HD8 (3.2 Fibril+HD8), although not to the same extent as with radiolabeled HD1.

Synthetic HD1 and HD8 were also tested for their ability to inhibit fibril formation in an in vitro Huntingtin aggregation assay. In this assay, Huntingtin protein aggregated over a period of 6 days, forming fibrils that could be imaged using electron microscopy (FIG. 8; Controls). Addition of either synthetic HD1 (top) or synthetic HD8 (bottom) resulted in significant inhibition of fibril formation in this assay, with little to no fibril formed after 6 days.

HD1 and HD8 binding was analyzed by electron paramagnetic resonance (EPR). The synthetic peptides were spin labeled using a free Cys at the C-terminus of the peptides. The free peptides and peptides bound to Htt-fibrils were analyzed by EPR and the data shown in FIG. 10. Upon addition of Htt fibrils, a decrease in central line amplitude is observed, indicating the formation of peptide-fibril complex. The stoichiometry of the complex was also studied using EPR, with varying amounts of peptide added to fibrils (FIG. 10, panel B). HD1 binding is saturated at a 1:3 ratio, whereas HD8 shows at least a 1:1 ratio.

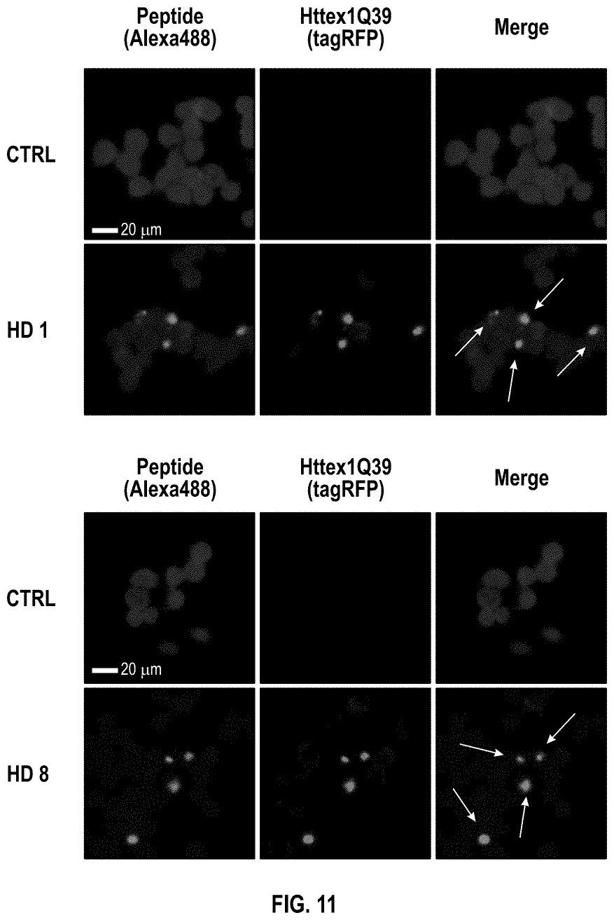

HD1 and HD8 were also tested to see if the synthetic peptide recognized Huntingtin fibrils from cells. In this assay, HD1 and HD8 are were labeled with the fluorescent dye, Alexa 488 and were incubated with fixed N2A cells (a mouse neuroblastoma cell line) that were expressing Huntingtin Exon 1 Q39 (mutant Huntingtin with 39 CAG repeats) fused to red fluorescent protein (tagRFP). In control experiments, with no Huntingtin exon 1 (Q39)-RFP fusion (Httex1 Q39) transfected, a diffuse signal is observed with HD1 and HD8 peptides (FIG. 11; Controls). When Httex1 Q39 is transfected and probed with Alexa488 HD1 or HD8, significant colocalization of Alexa 488 signal (FIG. 11; pictured in the first column of HD1 and HD8 row of images) and RFP signal (FIG. 11; pictured in the second column of HD1 and HD8 row of images) is observed (FIG. 11; indicated by arrows in the third column of HD1 and HD8 row of images *), indicating an interaction between the HD1 or HD8 peptide and the Httex1Q39 protein (presumably, aggregated unbundled fibrils).

### Example 4. Mutational Analysis of HD1 and HD8

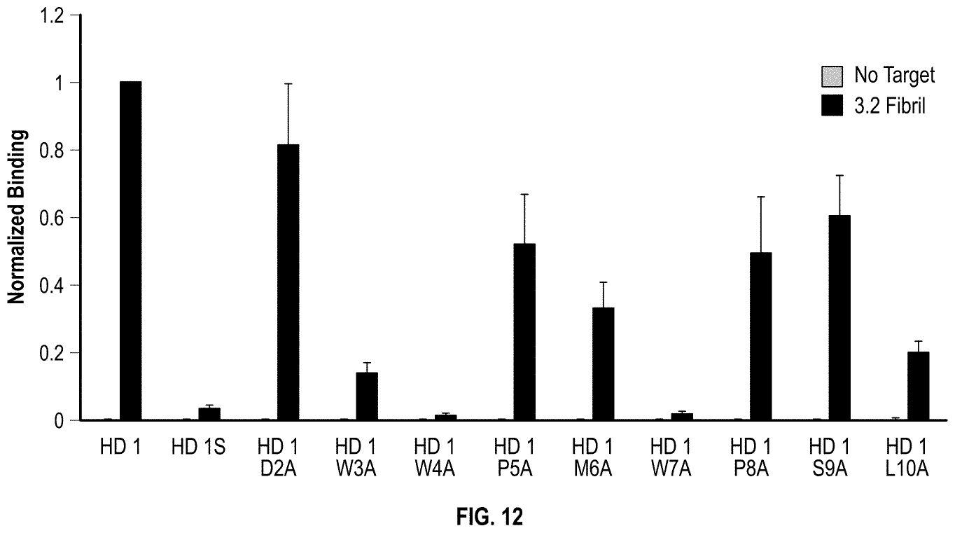

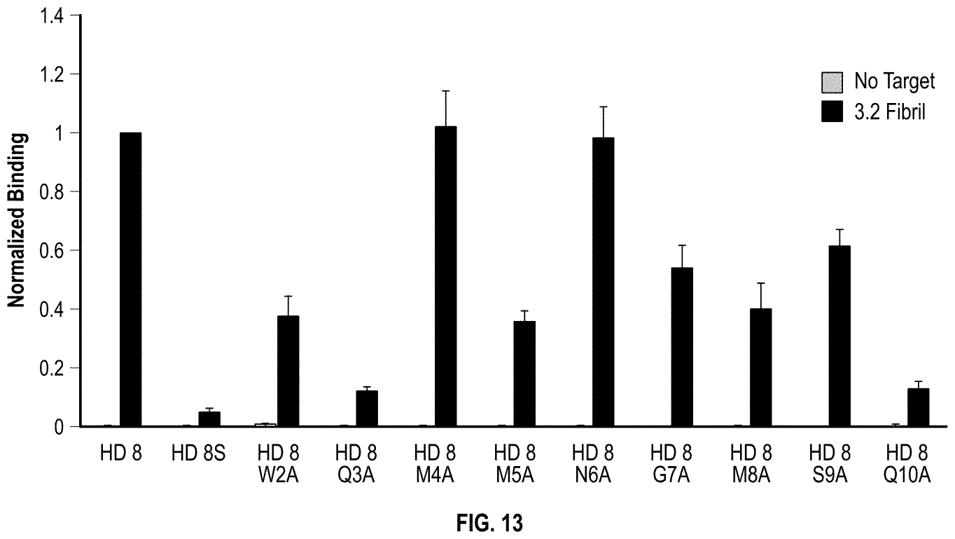

To determine what residues of HD1 and HD8 are critical for binding, we performed an Alanine scan of the peptides (Cunningham, et al. (1989) Science 244: 1081-5). We thus systematically constructed and tested every positional mutant where each wild type residue of HD1 and HD8 were mutated to Alanine using our radioactive binding assay described above. The data show that substitution of Ala at W4, and W7 virtually abolish all binding, indicating that these positions are extremely important for binding of HD1 (FIG. 12). Mutations of W3, P5, M6, P8, S9, and L10 all decrease binding, indicating that these positions contribute to HD1 binding. Similarly, for HD8, positions Q3 and Q10 show large losses of binding when mutated to Ala, with modest losses for W2, M5, G7, M8, and S9, all indicating that these positions are important (Q3 and Q10) or contribute (W2, M5, G7, M8, and S9) to HD8 binding.

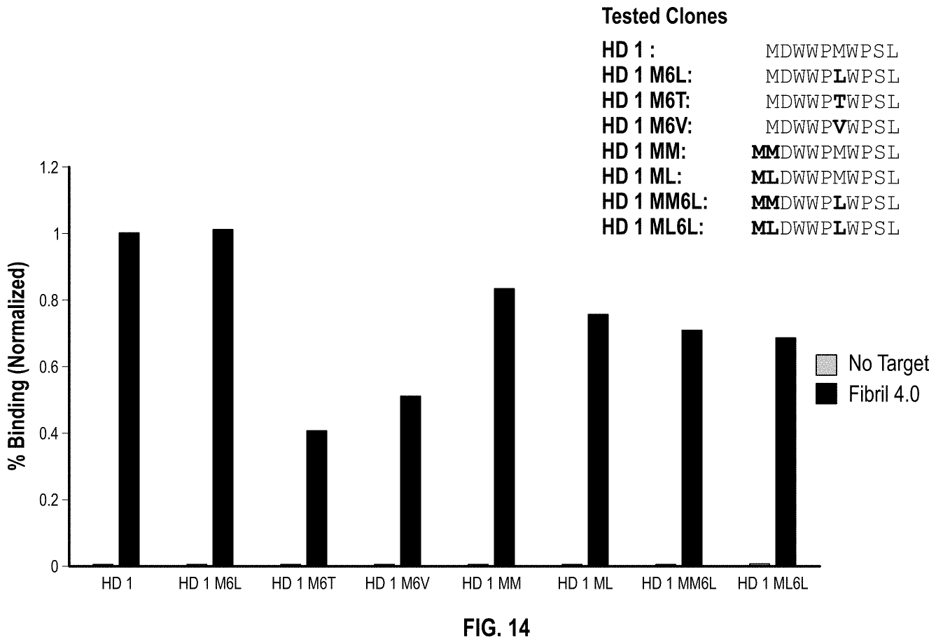

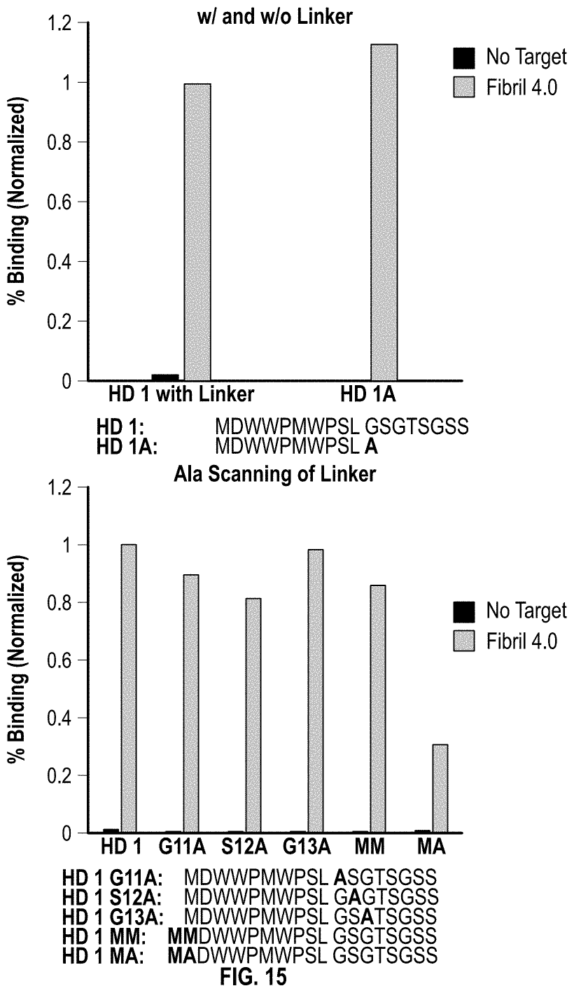

Alanine scanning of HD1 indicated that substitution of M6 with Ala reduced but did not abolish HD1 binding. In peptides used for therapeutic purposes, the presence of sulfur in the peptides is undesirable because of oxidation, thereby changing the peptide. Thus, it is desirable to remove sulfur containing amino acids, such as Cys or Met, from a peptide used for therapeutic purposes. We thus performed additional mutational experiments on HD1 attempting to test if either M1 or M6 could be substituted for a different amino acid. We thus designed a series of mutants that changed M6 to Leu, Thr, or Val. We also aimed to do a similar experiment with Ml, however, since virtually all proteins translated by the ribosome begin with Met, we could not easily substitute M1 for another amino acid, as Met at position 1 is needed to initiate translation. Instead, for M1 mutational analysis, we first added a second Met to the N-terminus, making the peptide sequence MMDWWPMWPSL (HD1MM). We then were able to substitute M1 with Leu, since the Met added at the N-terminus would be used for translation initiation. FIG. 14 shows the results of these data—most substitutions of Met with the exception of M6T and M6V showed either no effect on binding (HD1 M6L) or a slightly reduced (20-25%) binding (HD1 MM, HD1 ML, HD1 MM6L, and ML6L). These results suggest that both M1 and M6 could be substituted with a hydrophobic, non-sulfur containing amino acid. It is likely that other non-natural hydrophobic, non-sulfur containing amino acids could likely be substituted (e.g., norleucine or norvaline).

We also tested the effect of the C-terminal linker that is kept constant and is necessary for mRNA display selections. Using our radioactive binding assay described above, we tested to see if removal of the C-terminal GSGTSGSS constant region used in the mRNA display selection against Huntingtin fibrils would have an effect on HD1 binding. FIG. 15 shows that substitution of the GSGTSGSS constant region with Ala (HD 1A) has little effect on binding affinity versus HD1 containing the GSGTSGSS constant region. Similarly, we performed an Alanine scanning experiment to see if mutation of the amino acids in the GSGTSGSS constant region had an effect on binding (FIG. 15). Mutation of G11, S12, or G13 all had little effect (<20% decrease) in HD1 binding, suggesting that these positions were not important for high affinity HD1 binding. We also performed an Alanine mutation of the previously described peptide with an additional Met at the N-terminus, changing Met1 to Ala (HD1 MA). Comparing this mutant (HD1 MA) to the Met extended peptide (HD1 MM) shows that mutation of M1 to Ala significantly decreases binding. These data coupled with the Leucine substitution data above for M1 thus suggest that Met1 can be substituted with a larger, longer, hydrophobic amino acid.

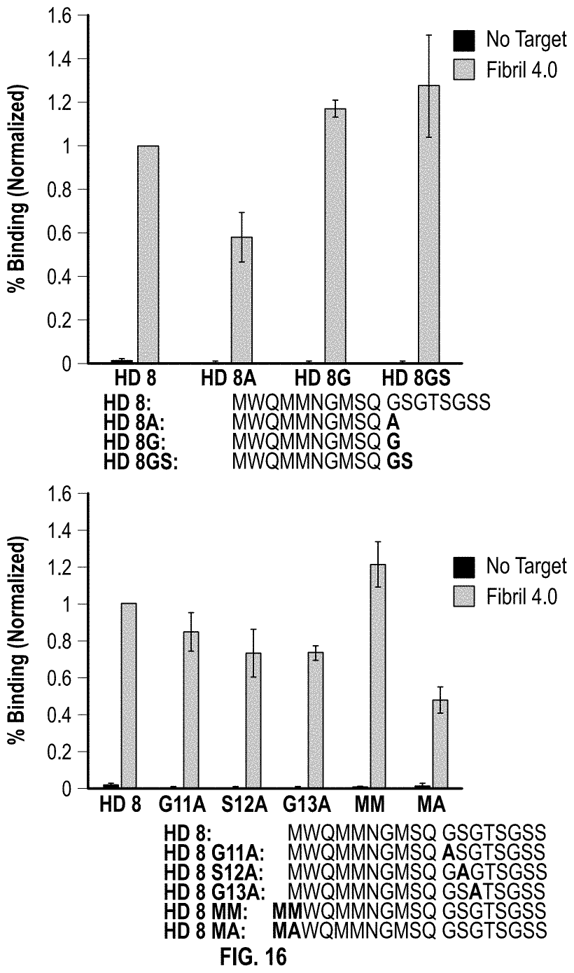

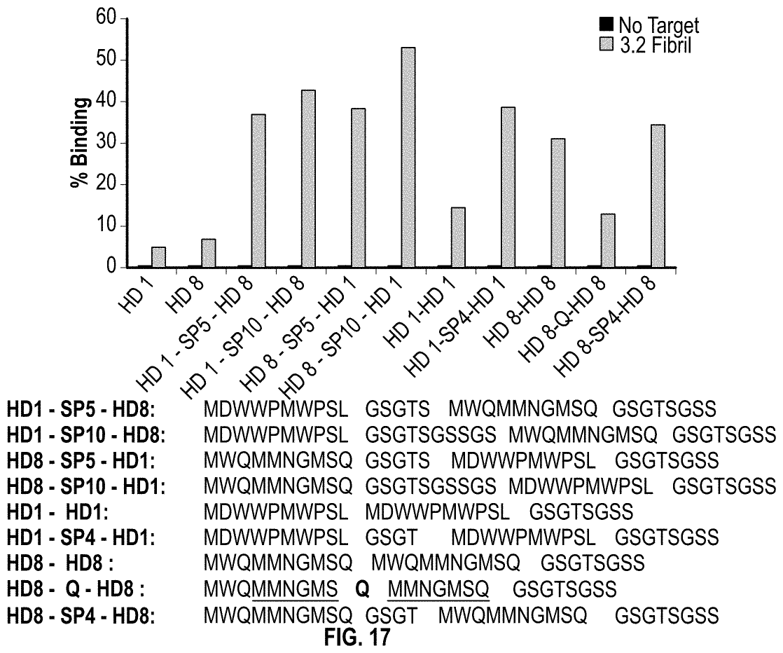

Mutational analysis on the HD8 constant region was also performed. First, the GSGTSGSS constant region of HD8 was removed and substituted with Ala (HD 8A), which showed a modest loss in binding affinity. This loss could be recovered by substituting the GSGTSGSS constant region with Gly (HD 8G) or by Gly-Ser (HD 8GS) as shown in FIG. 16. Mutation of G11, S12, and G13 to Ala also resulted in modest decreases in HD8 binding affinity. Similarly, comparing the N-terminal Met extension sequence (HD8 MM) to the M1 Ala mutant (HD8 MA) also showed a decrease in binding of M1. The conclusion of these data are that Gly11 could be important for HD8 binding while M1 seems to be important in contributing to HD8 binding affinity.

### Example 5. Multimerization of Huntingtin-Binding Peptide Agents

The fibrils formed from the aggregation of Huntingtin would suggest a fibril structure that had repeating structural elements. While one skilled in the art can recognize that reports in the literature have noted increases in affinity due to avidity effects (Vauquelin, et al. (2013) Br J Pharmacol 168: 1771-85), however avidity effects are not always achieved by simple multimerization. We hypothesized that due to the repeating structure of fibrils, multimerization of a fibril binding peptide agent would likely result in a large increase in binding affinity in a general and reproducible way.

To test this hypothesis, we first constructed a number of dimeric sequences of the form (peptide agent)-spacer-(peptide agent) or (peptide agent)-(peptide agent). The spacer was composed of a mixture of Gly, Ser, or Thr, in order to provide flexibility (Gly) or water solubility (Ser or Thr). The spacers used were also varied between 5 amino acids or 10 amino acids, though either shorter, in between, or longer spacers could be used. The data in FIG. 17 show that the binding of HD1 and HD8 (left two samples) can be increased by dimerizing the two peptides. Both HD1 and HD8 were used as peptide agents and were used either as heterodimeric pairs (containing both HD1 and HD8) or homodimeric pairs (containing only HD1 or only HD8). All dimers show increased binding versus the monomeric HD1 or HD8 peptide agents, supporting our overall hypothesis that the fibril provides excellent binding sites for multivalent peptide agents.

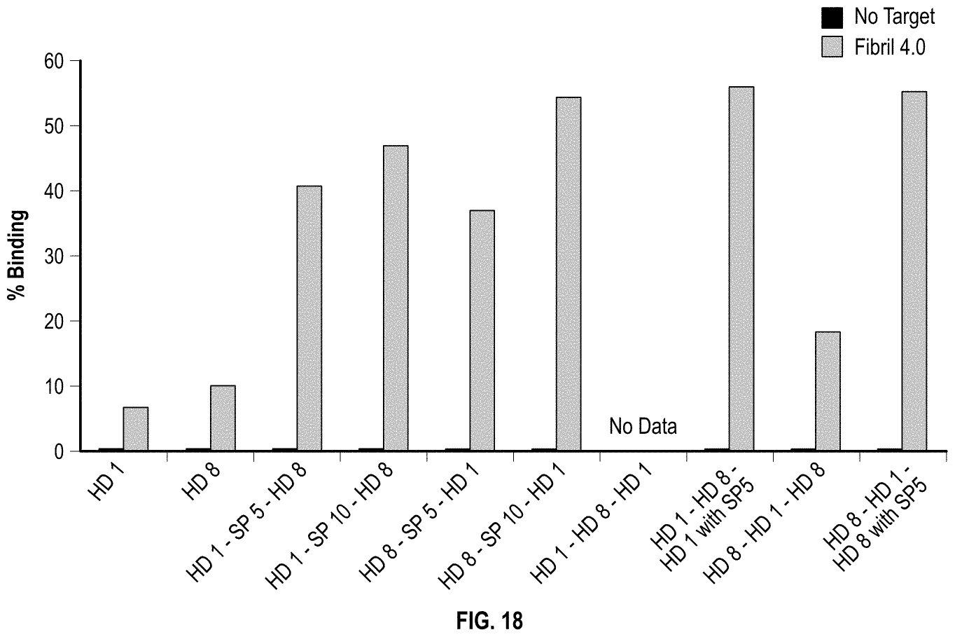

We further tested if extending this strategy would yield even higher affinity binders. To do this, we created trimeric (FIG. 18) peptide agents of the form (peptide agent)-spacer-(peptide agent)-spacer-(peptide agent) or (peptide agent)-(peptide agent)-(peptide agent) and tested these constructs using a radioactive binding assay described above. The data show that trimerizing the peptide agents yields even higher binding peptide agents as compared with the dimeric binding peptide agents (FIG. 18). However, we noticed that the trimeric peptide agents resulted in binders that gave 50-60% binding in the radioactive binding assay, and we hypothesized that the binding in this assay may have been saturated.

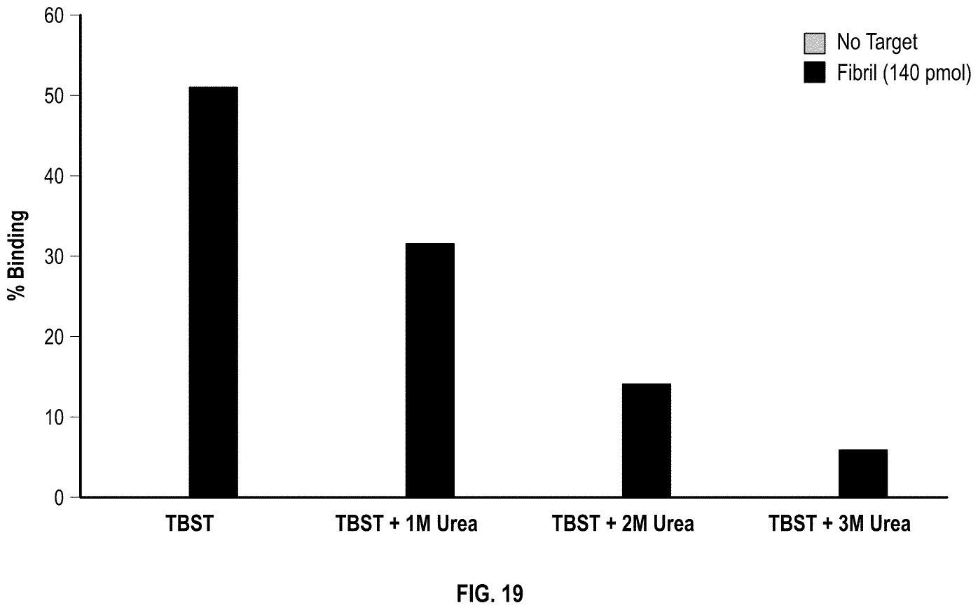

To test if the radioactive binding assay was saturated, we modified the binding assay to include more stringent binding conditions. We first tested if the presence of urea, a known denaturant of protein structure, would decrease overall binding in the assay. Using the dimeric HD8SP10-HD1 peptide agent, we tested if addition of 1 M, 2 M, or 3 M would result in decreased binding as compared to a buffer only (TBST) control (FIG. 19). The data show that indeed, addition of higher concentrations of urea resulted in decreased binding that is approximately proportional to the amount of urea added.

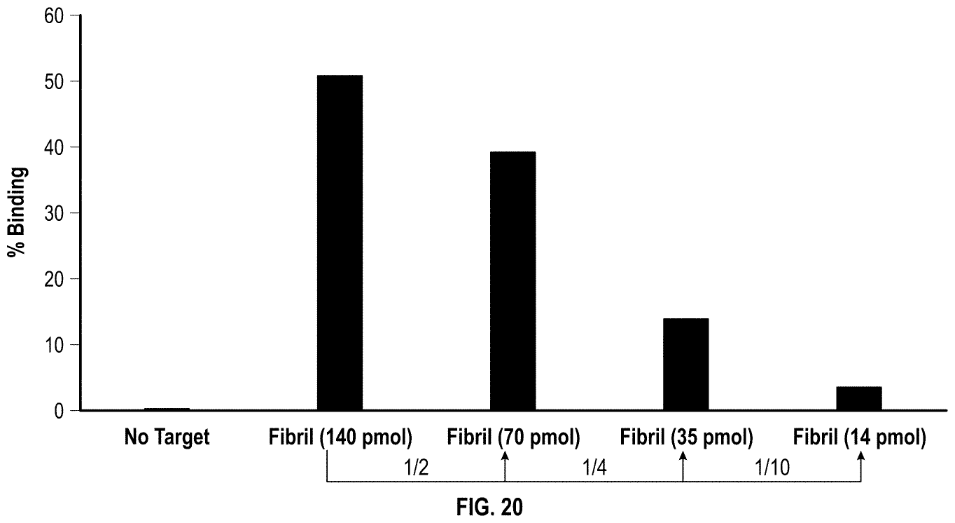

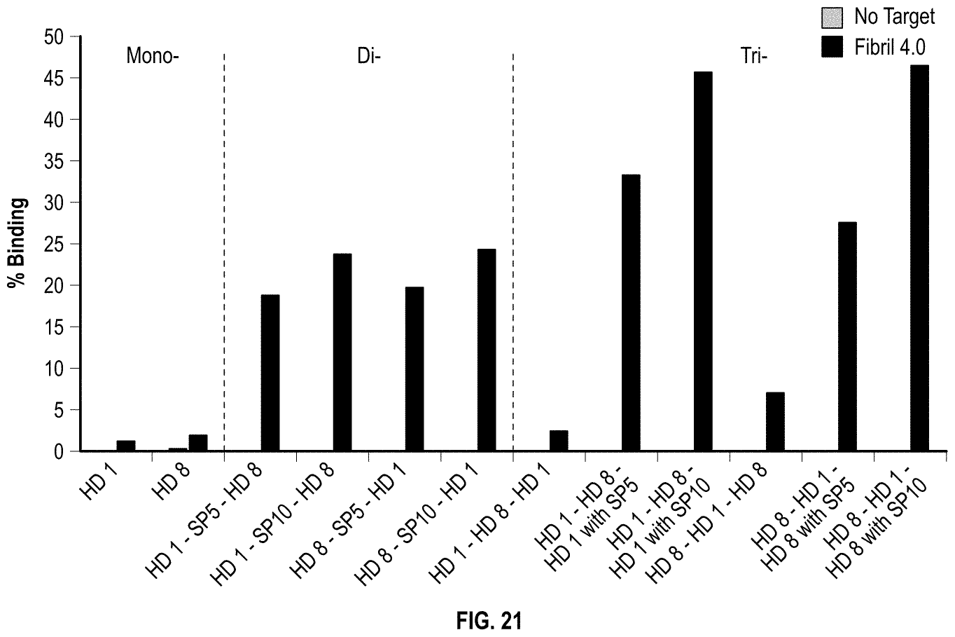

We also increased binding stringency in the radioactive binding assay by varying the amount of target that was immobilized. FIG. 20 shows the effect of decreasing the amount of fibril immobilized from 140 pmol to 14 pmol, resulting in a roughly proportional decrease in binding with decreased amounts of target.

We thus retested the monomeric, dimeric, and trimeric peptide agents using lower fibril conditions. We chose lower fibril conditions since less fibril would be consumed in the binding tests as well as the worry that the addition of urea might affect protein structure or the nature of the binding reaction. Under conditions where less fibril was immobilized, we observed a larger difference between the trimeric peptide agents. Although all trimers showed higher binding than the monomeric peptide agents, certain combinations were better performing, such as the HD1-SP10-HD8-SP10-HD1 peptide agent.

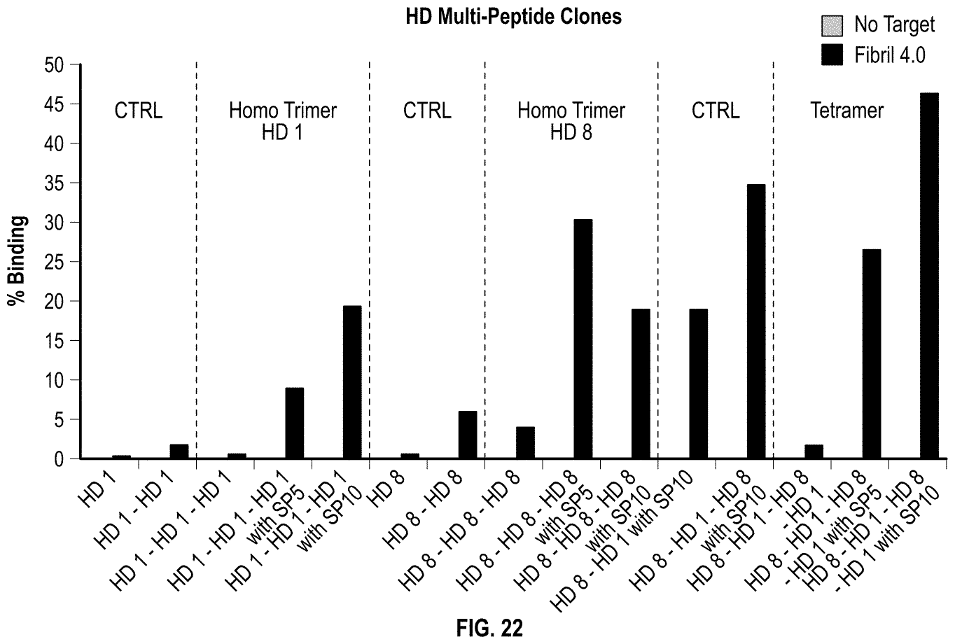

We then tested if tetramerizing peptide agents would yield even stronger binding peptide agents. We further decreased the amount of immobilized fibril and tested tetrameric peptide agents, which again resulted in even higher affinity peptide agents (FIG. 22).

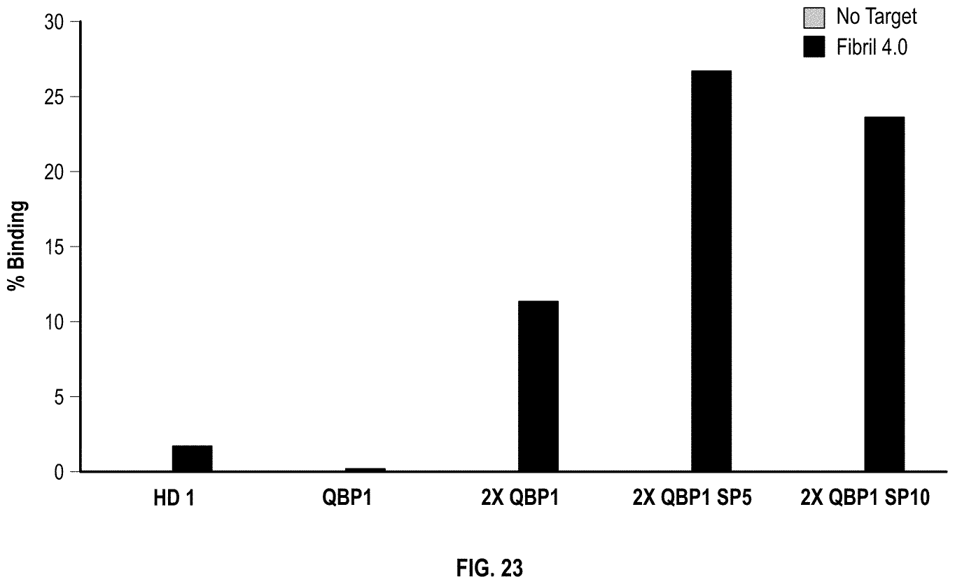

Lastly, to demonstrate that the enhancement of binding due to multimerization is not specific to the HD1 or HD8 peptides, we also tested if multimerization of the QBP1 peptide would result in increased binding. The data in FIG. 23 show the results of this experiment. We observe that multimerization of QBP1 does indeed increase binding, demonstrating that the multimerization strategy to increase binding is a general strategy.

Overall, the data show that multimerizing consistently and predictably increases binding over the monomeric peptide agents. While some optimization of the spacer length between peptide agents does seem to be important, in general, including a spacer seems to result in overall higher affinities.

### Example 6. Uses of Multimeric Huntingtin-Fibril Binding Peptide Agents

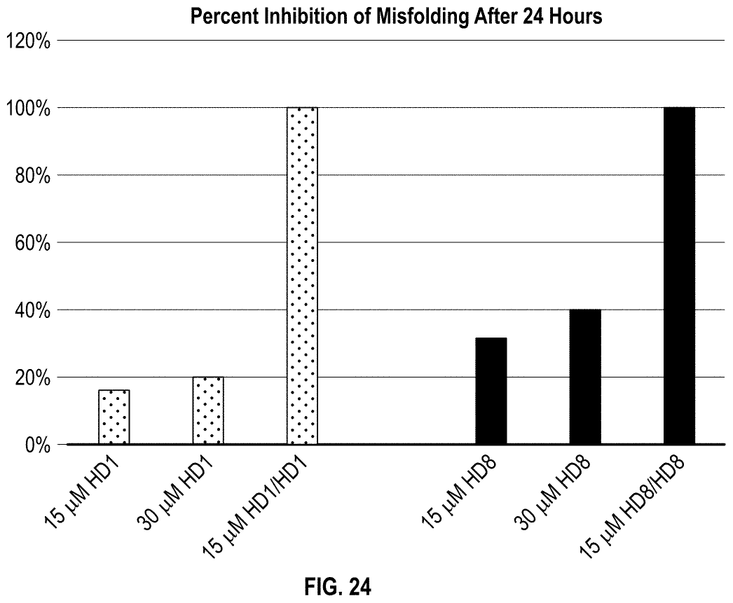

Increasing the affinity of peptide agents is generally a desirable goal as higher affinity peptide agents often have more utility than lower affinity peptide agents. We thus were interested in testing if the higher affinity peptides that we achieved by multimerization resulted in higher inhibition of fibril formation. Using the EPR to monitor the amount of aggregation, we tested to see how multimeric peptide agents compared with monomeric peptide agents in inhibiting fibril formation. FIG. 24 shows the percent inhibition of fibril formation under seeded conditions of monomeric HD1 and HD8, as compared with homodimeric HD1 and HD8. Under seeded conditions, fibrils form quickly and long complete aggregation within 24 h. Addition of HD1 and HD8 under these seeded conditions can result in up to ˜40% inhibition. This effect is on par with the best fibril-directed inhibiting peptide agents as judged by EPR spectroscopy, ThT monitored fibril formation, and electron microscopy (data not shown). This observation is exciting because the peptides block some specific step during the aggregation process. Remarkably, the aggregation inhibition of homodimeric peptides (HD1/HD1 or HD8/HD8) was much stronger with no detectable aggregation after 24 h (FIG. 24). Some small amount of aggregation was observed after 5 days of incubation with homodimeric peptides, but 80% of Httex1 remained monomeric (data not shown). Even at the present early stages of peptide design, the inhibition is unusually strong, and it represents the most potent fibril inhibitor ever tested in our hands for any amyloid fibril.

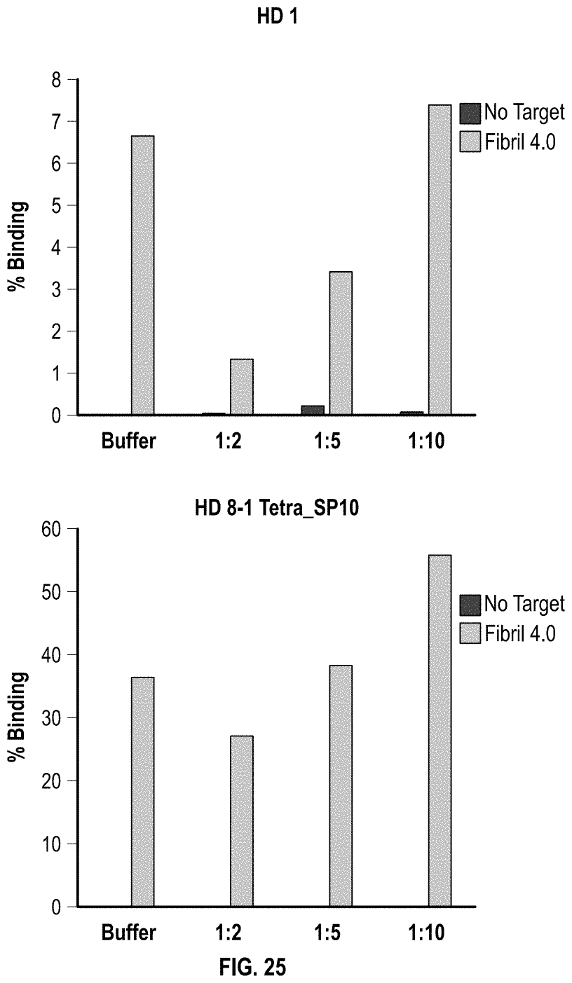

In a second experiment, we tested the ability of our peptides to bind Htt-fibrils in the presence of serum. HD1 was tested in buffer alone or with a 1:2, 1:5, or 1:10 dilution of serum with buffer (FIG. 25; top panel). The data show that serum partially inhibits the binding of HD1, however binding can be recovered when serum is diluted with buffer. In another experiment, the tetrameric HD8-SP10-HD1-SP10-HD8-SP10-HD1 molecule was tested in a similar fashion, and similar results were observed; serum can partially inhibit binding, however binding can be recovered with higher dilutions of serum with buffer. These results are encouraging since they suggest that the monomeric or multimeric fibril binding peptides could be used to recognize and detect the presence of fibrils in human serum.

### Example 7. HD8/HD1 Peptide Labelling of Cells and Tissues

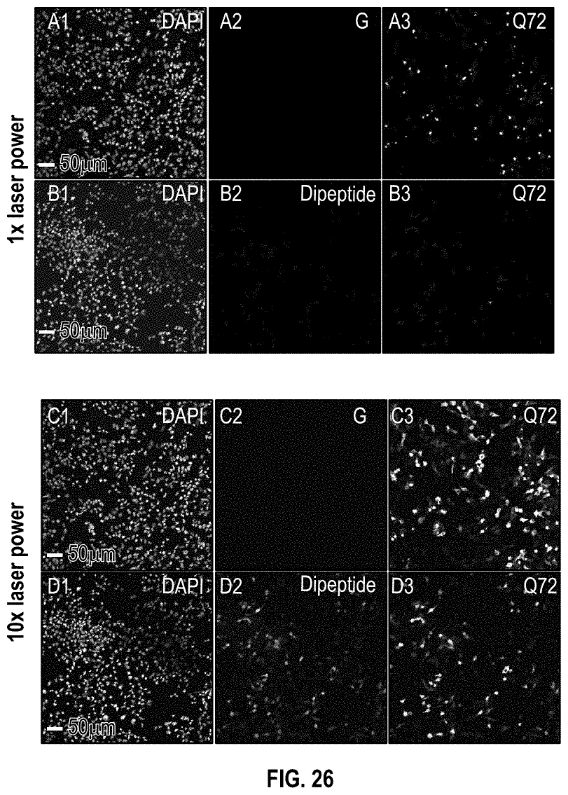

Building upon the in vitro evidence that peptides screened from the mRNA display bind strongly to sonicated unbundled fibrils, we tested their ability to affect the rate of Httex1 aggregate formation in cultured cells. Httex1 with an expanded Q length (Httex1Q72) readily forms aggregates in HEK293T cells 24 h after transfection. To visualize Httex1Q72 aggregates, a fluorescent tag, RFP, was appended to the carboxyl terminus of Httex1Q72. FIG. 26 shows an experiment to compare the amount of Httex1Q72RFP expressed in HEK293T cells that were transfected with the Httex1 construct alone (A and C), or together with GFP-HD8-SP10-HD1 (B and D). The total number of cells in each field was similar, as can be seen by nuclear stain (A1 to D1). The GFP signal from the HD peptides is shown in panels A2 to D2. No signal can be seen in A2 and C2 due to the absence of the construct. The Httex1 Q72 signal is shown in panels A3 to D3. Two different laser powers (1× and 10×) were used to capture a wider range of signal intensities. A comparison between A3 (without HD construct) and B3 (with HD construct) shows a decrease in HttexQ72 signal in A3. This can also be seen using 10× laser power (compare C3 and D3), which was used to capture weaker signals from GFP-HD8-SP10-HD1 and Httex1Q72-RFP. This laser setting is also used to demonstrate similar transfection efficiencies in both conditions (with or without HD peptides).

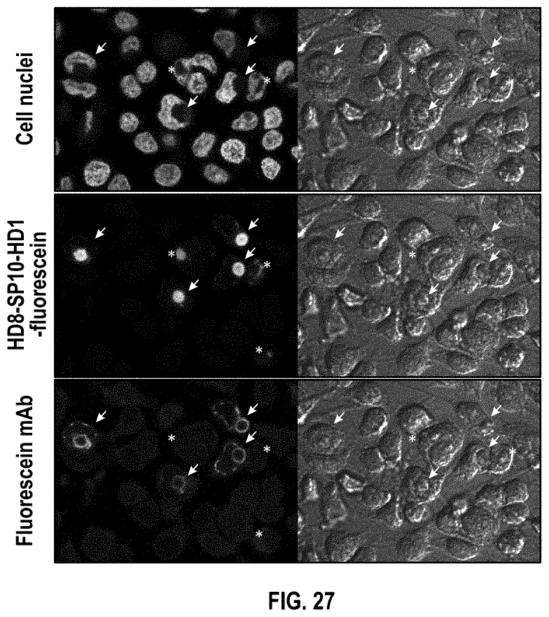

Next, we tested the ability of HD peptides to bind Httex1 aggregates formed in cultured cells. To do this, Httex1Q72 (without RFP) was transfected into HEK293 cells. After 24 hours, cells were fixed and incubated overnight with 0.1 micromolar of HD8-HD1 labeled with fluorescein at its carboxyl terminus (HD8-HD1-fluorescein. Without the RFP fluorescent tag, the Httex1Q72 aggregates can be visualized under phase contrast (FIG. 27, right panels). The total number of cells in the field is visualized with a nuclear stain (FIG. 27, top left). The fluorescein signal from the HD8-HD1 peptide is shown in the left middle panel. The result show strong labeling of the round cytoplasmic Httex1Q72 aggregates (arrows) and intranuclear aggregates (asterisks). To see whether the signal from the HD peptide can be further amplified using a mouse monoclonal antibody against fluorescein, the cells were also incubated with antibody, followed by a Alexa594-tagged secondary to distinguish this signal from fluorescein (bottom left panel). The antibody labeled the rim of the cytoplasmic aggregates and the signal also surrounded some cell nuclei. Little antibody staining was observed on intranuclear aggregates.

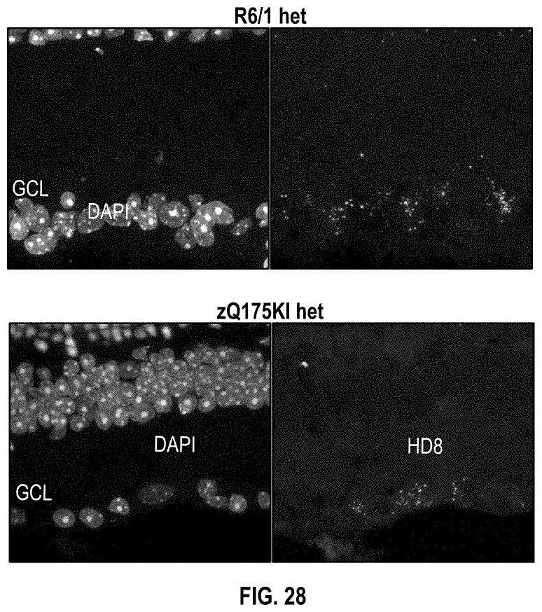

To see whether HD peptides bind Httex1 aggregates formed in vivo, we used tissues from two different Huntington mouse models, R6/1 (34 weeks old) and zQ175KI (48 weeks). FIG. 28 show retinal sections from these mice incubated with 2 micromolar of HD8 conjugated with Alexa488 at its carboxyl terminus. Cell nuclei is shown at the left panels, and signal from HD8, in the form of small puncta, was observed mostly in the ganglion cell layer (right panels).

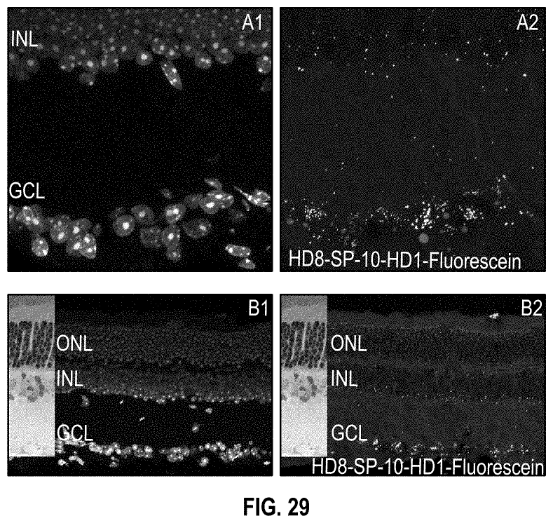

HD8-HD1-fluorescein (0.1 micromolar) was also used on retinal sections from 34-week-old R6/1 mice (FIG. 29). Robust signal from the peptide can be seen in the ganglion cell layer, again in the form of small puncta. In addition, weaker signal from intranuclear aggregates was also observed. Further, the signal can now be detected in all layers of the retina where the nuclei reside. These results are consistent with the higher efficacy of multimerized HD peptides in binding Httex1 aggregates.

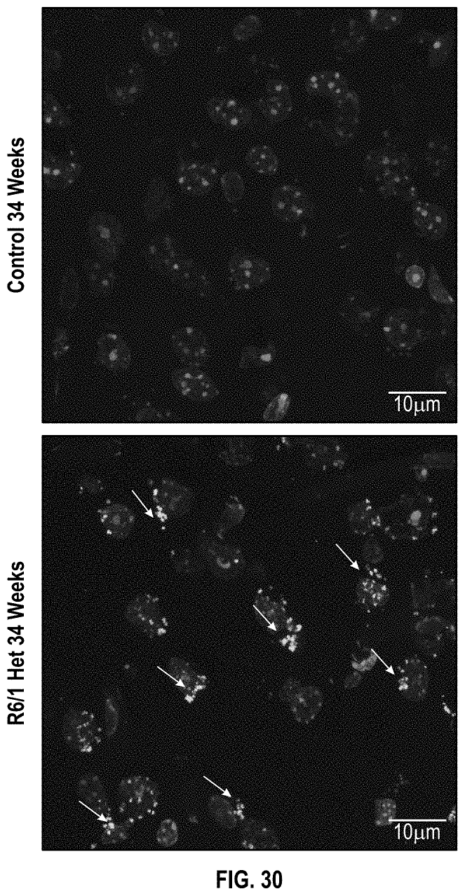

HD8-HD1-fluorescein was also used on brain sections from R6/1 mice (34 weeks). FIG. 30 show little signal from age-matched control mice (top panel), but abundant signal from R6/1 brain. Together, these results demonstrate the potential of the HD peptides as biomarker or therapeutic tools for Huntington disease.

### Example 8. Selected References

1. Walker, F. O., Huntington's disease. Lancet 369, 218-28 (2007).
2. Roberts, R. W. and Szostak, J. W., RNA-peptide fusions for the in vitro selection of peptides and proteins. Proc Natl Acad Sci USA 94, 12297-302 (1997).
3. Takahashi, T. T., et al., mRNA display: peptide agent discovery, interaction analysis and beyond. Trends Biochem Sci 28, 159-65 (2003).
4. Takahashi, T. T. and Roberts, R. W., In vitro selection of protein and peptide libraries using mRNA display. Methods Mol Biol 535, 293-314 (2009).
5. Jackson, R. J. and Hunt, T., Preparation and use of nuclease-treated rabbit reticulocyte lysates for the translation of eukaryotic messenger RNA. Methods Enzymol 96, 50-74 (1983).
6. Krejci, A., et al., Hammock: a hidden Markov model-based peptide clustering algorithm to identify protein-interaction consensus motifs in large datasets. Bioinformatics 32, 9-16 (2016).
7. Cetin, M., et al., RasIns: Genetically Encoded Intrabodies of Activated Ras Proteins. J Mol Biol 429, 562-573 (2017).
8. Jalali-Yazdi, F., et al., High-Throughput Measurement of Binding Kinetics by mRNA Display and Next-Generation Sequencing. Angew Chem Int Ed Engl 55, 4007-10 (2016).
9. Nagai, Y., et al., Inhibition of polyglutamine protein aggregation and cell death by novel peptides identified by phage display screening. J Biol Chem 275, 10437-42 (2000).
10. Cunningham, B. C. and Wells, J. A., High-resolution epitope mapping of hGH-receptor interactions by alanine-scanning mutagenesis. Science 244, 1081-5 (1989).
11. Vauquelin, G. and Charlton, S. J., Exploring avidity: understanding the potential gains in functional affinity and target residence time of bivalent and heterobivalent peptide agents. Br J Pharmacol 168, 1771-85 (2013).

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features described herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally described herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless described herein.

The technology illustratively described herein suitably can be practiced in the absence of any element(s) not specifically described herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

1. A peptide agent comprising one or more peptides comprising an amino acid sequence having at least 70% identity to an amino acid sequence selected from:
(i)
(HD1; SEQ ID NO: 1)
MDWWPMWPSL;
(ii)
(HD2; SEQ ID NO: 2)
MWMIQMPGYQ;
(iii)
(HD3; SEQ ID NO: 3)
MRWSMSYSWA;
(iv)
(HD4; SEQ ID NO: 4)
MFMMMWMSLT;
(v)
(HD5; SEQ ID NO: 5)
MFFVLSWTPL;
(vi)
(HD6; SEQ ID NO: 6)
MQMWTMWEPW;
(vii)
(HD7; SEQ ID NO: 7)
MDLWPMWESW;
(viii)
(HD8; SEQ ID NO: 8)
MWQMMNGMSQ;
(ix)
(HD1-Derivative; SEQ ID NO: 9)
MX₂X₃X₄X₅X₆X₇X₈X₉X₁₀,
wherein X₂ is selected from D, H, L, N, T and Y; X₃ is selected from F, R and W; X₄ is selected from R and W; X₅ is selected from H, P, S and T; X₆ is selected from L, M, Q, S, T and V; X₇ is selected from R and W; X₈ is selected from A, E, H, P, R, S, and T; X₉ is selected from D, P, Q, S, T and W; and X₁₀ is selected from L, M, P, R and T;
(x) MX₂X₃WX₅X₆WX₈X₉X₁₀ (HD6/7-Derivative; SEQ ID NO:10), wherein X₂ is selected from A, D, E, I, L, M, N, P, Q, T and V; X₃ is selected from F, L, M, S, W and Y; X₅ is selected from A, D, E, G, N, P, Q and T; X₆ is selected from F, L, M and W; X₈ is selected from D, E, N, Q and P; X₉ is selected from C, D, N, P, S and T; and X₁₀ is selected from L and W;
(xii) MWX₃X₄X₅X₆X₇X₈X₉Q (HD2-Derivative; SEQ ID NO:11), wherein X₃ is selected from M and Q; X₄ is selected from I and M; X₅ is selected from M and Q; X₆ is selected from F and M; X₇ is selected from I, M, N, P and Q; X₈ is selected from F, G, M, S and W; and X₉ is selected from E and Y; and
(xiii) MX₂QX₄MX₆X₇X₈X₉Q (HD8-Derivative; SEQ ID NO:12), wherein X₂ is selected from F, W and Y; X₄ is selected from L, M, W and Y; X₆ is selected from G, H, N, S and Y; X₇ is selected from G and N; X₈ is selected from I and M; and X₉ is selected from G, S and Y.

2. The peptide agent of claim 1, wherein the one or more peptides comprise one or more conservative amino acid substitutions.

3. The peptide agent of claim 1, wherein the one or more peptides comprise of an amino acid sequence selected from MAWWPMWPSL (HD1-D2A; SEQ ID NO:13), MDAWPMWPSL (HD1-W3A; SEQ ID NO:14), MDWWAMWPSL (HD1-P5A; SEQ ID NO:15), MDWWPAWPSL (HD1-M6A; SEQ ID NO:16), MDWWPMWASL (HD1-P8A; SEQ ID NO:17), MDWWPMWPAL (HD1-S9A; SEQ ID NO:18), and MDWWPMWPSA (HD1-L10A; SEQ ID NO:19).

4. The peptide agent of claim 1, wherein the one or more peptides comprise an amino acid sequence selected from MDWWPLWPSL (HD1-M6L; SEQ ID NO:20), MDWWPTWPSL (HD1-M6T; SEQ ID NO:21), MDWWPVWPSL (HD1-M6V; SEQ ID NO:22), MMDWWPMWPSL (HD1-MM; SEQ ID NO:23), MLDWWPMWPSL (HD1-ML; SEQ ID NO:24), MMDWWPLWPSL (HD1-MM6L; SEQ ID NO:25), and MLDWWPLWPSL (HD1-ML6L; SEQ ID NO:26).

5. The peptide agent of claim 1, wherein the one or more peptides comprise an amino acid sequence selected from MAQMMNGMSQ (HD8-W2A; SEQ ID NO:27), MWAMMNGMSQ (HD8-Q3A; SEQ ID NO:28), MWQAMNGMSQ (HD8-M4A; SEQ ID NO:29), MWQMANGMSQ (HD8-MSA; SEQ ID NO:30), MWQMMAGMSQ (HD8-N6A; SEQ ID NO:31), MWQMMNAMSQ (HD8-G7A; SEQ ID NO:32), MWQMMNGASQ (HD8-M8A; SEQ ID NO:33), and MWQMMNGMAQ (HD8-S9A; SEQ ID NO:34).

6. The peptide agent of claim 1, comprising two, three or four of the one or more peptides.

7. The peptide agent of claim 1, further comprising one or more linkers.

8. The peptide agent of claim 7, wherein the linker comprises or consists of an amino acid selected from A, G and S, or an amino acid sequence selected from GS, GT, GSG, GSGT (SEQ ID NO:47) and GSGTS (SEQ ID NO:48).

9. The peptide agent of claim 7, wherein the linker comprises an amino acid sequence selected from GSGTSGSS (SEQ ID NO:35), ASGTSGSS (SEQ ID NO:36), GAGTSGSS (SEQ ID NO:37), GSATSGSS (SEQ ID NO:38) and GSGTSGSSGS (SEQ ID NO:50).

10. The peptide agent of claim 1, wherein the one or more peptides comprise an amino acid sequence selected from MWQMMNGMSQ GSGTSGSS (SEQ ID NO:39); MWQMMNGMSQA (HD8A; SEQ ID NO:40); MWQMMNGMSQG (HD8G; SEQ ID NO:41); MWQMMNGMSQGS (HD8-GS; SEQ ID NO:42); MWQMMNGMSQ ASGTSGSS (HD8-G11A; SEQ ID NO:43); MWQMMNGMSQ GAGTSGSS (HD8-G13A; SEQ ID NO:44); MMWQMMNGMSQ GSGTSGSS (HD8MM;
SEQ ID NO:45) and MAWQMMNGMSQ GSGTSGSS (HD8MA; SEQ ID NO:46).

11. The peptide agent of claim 7, comprising two or more of the one or more peptides connected by the one or more linkers.

12. (canceled)

13. The peptide agent of claim 1, comprising the amino acid sequence of:
[(SEQ ID NO:1)-(SEQ ID NO:48)-(SEQ ID NO:2)-(SEQ ID NO:35)](HD1-SP5-HD8);
[(SEQ ID NO:1)-(SEQ ID NO:50)-(SEQ ID NO:2)-(SEQ ID NO:35)](HD1-SP10-HD8);
[(SEQ ID NO:2)-(SEQ ID NO:48)-(SEQ ID NO:1)-(SEQ ID NO:35)](HD8-SP5-HD1);
[(SEQ ID NO:2)-(SEQ ID NO:50)-(SEQ ID NO:1)-(SEQ ID NO:35)](HD8-SP10-HD1);
[(SEQ ID NO:1)-(SEQ ID NO:1)-(SEQ ID NO:35)](HD1-HD1);
[(SEQ ID NO:1)-(SEQ ID NO:47)-(SEQ ID NO:1)-(SEQ ID NO:35)](HD1-SP4-HD1);
[(SEQ ID NO:2)-(SEQ ID NO:2)-(SEQ ID NO:35)](HD8-HD8);
[(SEQ ID NO:2)-(MMNGMSQ (SEQ ID NO:49))-(SEQ ID NO:35)](HD8-Q-HD8);
[(SEQ ID NO:2)-(SEQ ID NO:47)-(SEQ ID NO:2)-(SEQ ID NO:35)](HD8-SP4-HD8); or
[(SEQ ID NO:2)-(SEQ ID NO:50)-(SEQ ID NO:1)-(SEQ ID NO:50)-(SEQ ID NO:2)-(SEQ ID NO:50)-(SEQ ID NO:1)](HD8-SP10-HD1-SP10-HD8-SP10-HD1).

14. The peptide agent of claim 1, wherein the one or more peptides have a length of 100 amino acids or less.

15. (canceled)

16. (canceled)

17. (canceled)

18. (canceled)

19. A method of preventing, slowing the onset or progression of, or treating Huntington's disease in a subject in need thereof comprising administering a pharmaceutical composition comprising a therapeutically effective amount of a peptide agent, wherein the peptide agent comprises one or more peptides comprising an amino acid sequence having at least 70% identity to an amino acid sequence selected from:
(i)
(HD1; SEQ ID NO: 1)
MDWWPMWPSL;
(ii)
(HD2; SEQ ID NO: 2)
MWMIQMPGYQ;
(iii)
(HD3; SEQ ID NO: 3)
MRWSMSYSWA;
(iv)
(HD4; SEQ ID NO: 4)
MFMMMWMSLT;
(v)
(HD5; SEQ ID NO: 5)
MFFVLSWTPL;
(vi)
(HD6; SEQ ID NO: 6)
MQMWTMWEPW;
(vii)
(HD7; SEQ ID NO: 7)
MDLWPMWESW;
(viii)
(HD8; SEQ ID NO: 8)
MWQMMNGMSQ;
(ix)
(HD1-Derivative; SEQ ID NO: 9)
MX₂X₃X₄X₅X₆X₇X₈X₉X₁₀,
wherein X₂ is selected from D, H, L, N, T and Y; X₃ is selected from F, R and W; X₄ is selected from R and W; X₅ is selected from H, P, S and T; X₆ is selected from L, M, Q, S, T and V; X₇ is selected from R and W; X₈ is selected from A, E, H, P, R, S, and T; X₉ is selected from D, P, Q, S, T and W; and X₁₀ is selected from L, M, P, R and T;
(x) MX₂X₃WX₅X₆WX₈X₉X₁₀ (HD6/7-Derivative; SEQ ID NO:10), wherein X₂ is selected from A, D, E, I, L, M, N, P, Q, T and V; X₃ is selected from F, L, M, S, W and Y; X₅ is selected from A, D, E, G, N, P, Q and T; X₆ is selected from F, L, M and W; X₈ is selected from D, E, N, Q and P; X₉ is selected from C, D, N, P, S and T; and X₁₀ is selected from L and W;
(xii) MWX₃X₄X₅X₆X₇X₈X₉Q (HD2-Derivative; SEQ ID NO:11), wherein X₃ is selected from M and Q; X₄ is selected from I and M; X₅ is selected from M and Q; X₆ is selected from F and M; X₇ is selected from I, M, N, P and Q; X₈ is selected from F, G, M, S and W; and X₉ is selected from E and Y; and
(xiii) MX₂QX₄MX₆X₇X₈X₉Q (HD8-Derivative; SEQ ID NO:12), wherein X₂ is selected from F, W and Y; X₄ is selected from L, M, W and Y; X₆ is selected from G, H, N, S and Y; X₇ is selected from G and N; X₈ is selected from I and M; and X₉ is selected from G, S and Y.

20. (canceled)

21. A method of preventing, inhibiting, mitigating, delaying, or reducing aggregation of Huntingtin protein or formation of Huntingtin fibrils in a subject in need thereof comprising administering a therapeutically effective amount of a peptide agent, wherein the peptide agent comprises one or more peptides comprising an amino acid sequence having at least 70% identity to an amino acid sequence selected from:
(i)
(HD1; SEQ ID NO: 1)
MDWWPMWPSL;
(ii)
(HD2; SEQ ID NO: 2)
MWMIQMPGYQ;
(iii)
(HD3; SEQ ID NO: 3)
MRWSMSYSWA;
(iv)
(HD4; SEQ ID NO: 4)
MFMMMWMSLT;
(v)
(HD5; SEQ ID NO: 5)
MFFVLSWTPL;
(vi)
(HD6; SEQ ID NO: 6)
MQMWTMWEPW;
(vii)
(HD7; SEQ ID NO: 7)
MDLWPMWESW;
(viii)
(HD8; SEQ ID NO: 8)
MWQMMNGMSQ;
(ix)
(HD1-Derivative; SEQ ID NO: 9)
MX₂X₃X₄X₅X₆X₇X₈X₉X₁₀,
wherein X₂ is selected from D, H, L, N, T and Y; X₃ is selected from F, R and W; X₄ is selected from R and W; X₅ is selected from H, P, S and T; X₆ is selected from L, M, Q, S, T and V; X₇ is selected from R and W; X₈ is selected from A, E, H, P, R, S, and T; X₉ is selected from D, P, Q, S, T and W; and X₁₀ is selected from L, M, P, R and T;
(x) MX₂X₃WX₅X₆WX₈X₉X₁₀ (HD6/7-Derivative; SEQ ID NO:10), wherein X₂ is selected from A, D, E, I, L, M, N, P, Q, T and V; X₃ is selected from F, L, M, S, W and Y; X₅ is selected from A, D, E, G, N, P, Q and T; X₆ is selected from F, L, M and W; X₈ is selected from D, E, N, Q and P; X₉ is selected from C, D, N, P, S and T; and X₁₀ is selected from L and W; (xii) MWX₃X₄X₅X₆X₇X₈X₉Q (HD2-Derivative; SEQ ID NO:11), wherein X₃ is selected from M and Q; X₄ is selected from I and M; X₅ is selected from M and Q; X₆ is selected from F and M; X₇ is selected from I, M, N, P and Q; X₈ is selected from F, G, M, S and W; and X₉ is selected from E and Y; and
(xiii) MX₂QX₄MX₆X₇X₈X₉Q (HD8-Derivative; SEQ ID NO:12), wherein X₂ is selected from F, W and Y; X₄ is selected from L, M, W and Y; X₆ is selected from G, H, N, S and Y; X₇ is selected from G and N; X₈ is selected from I and M; and X₉ is selected from G, S and Y.

22. A method of screening for a compound that binds to Huntingtin fibrils comprising (i) contacting a Huntingtin fibril with a peptide agent, thereby forming a complex, wherein the peptide agent comprises one or more peptides comprising an amino acid sequence having at least 70% identity to an amino acid sequence selected from:
(i)
(HD1; SEQ ID NO: 1)
MDWWPMWPSL;
(ii)
(HD2; SEQ ID NO: 2)
MWMIQMPGYQ;
(iii)
(HD3; SEQ ID NO: 3)
MRWSMSYSWA;
(iv)
(HD4; SEQ ID NO: 4)
MFMMMWMSLT;
(v)
(HD5; SEQ ID NO: 5)
MFFVLSWTPL;
(vi)
(HD6; SEQ ID NO: 6)
MQMWTMWEPW;
(vii)
(HD7; SEQ ID NO: 7)
MDLWPMWESW;
(viii)
(HD8; SEQ ID NO: 8)
MWQMMNGMSQ;
(ix)
(HD1-Derivative; SEQ ID NO: 9)
MX₂X₃X₄X₅X₆X₇X₈X₉X₁₀,
wherein X₂ is selected from D, H, L, N, T and Y; X₃ is selected from F, R and W; X₄ is selected from R and W; X₅ is selected from H, P, S and T; X₆ is selected from L, M, Q, S, T and V; X₇ is selected from R and W; X₈ is selected from A, E, H, P, R, S, and T; X₉ is selected from D, P, Q, S, T and W; and X₁₀ is selected from L, M, P, R and T;
(x) MX₂X₃WX₅X₆WX₈X₉X₁₀ (HD6/7-Derivative; SEQ ID NO:10), wherein X₂ is selected from A, D, E, I, L, M, N, P, Q, T and V; X₃ is selected from F, L, M, S, W and Y; X₅ is selected from A, D, E, G, N, P, Q and T; X₆ is selected from F, L, M and W; X₈ is selected from D, E, N, Q and P; X₉ is selected from C, D, N, P, S and T; and X₁₀ is selected from L and W.
(xii) MWX₃X₄X₅X₆X₇X₈X₉Q (HD2-Derivative; SEQ ID NO:11), wherein X₃ is selected from M and Q; X₄ is selected from I and M; X₅ is selected from M and Q; X₆ is selected from F and M; X₇ is selected from I, M, N, P and Q; X₈ is selected from F, G, M, S and W; and X₉ is selected from E and Y; and
(xiii) MX₂QX₄MX₆X₇X₈X₉Q (HD8-Derivative; SEQ ID NO:12), wherein X₂ is selected from F, W and Y; X₄ is selected from L, M, W and Y; X₆ is selected from G, H, N, S and Y; X₇ is selected from G and N; X₈ is selected from I and M; and X₉ is selected from G, S and Y;
(ii) contacting the complex with a test compound, and (iii) determining the presence or absence of a dissociated complex, wherein the presence of a dissociated complex indicates the test compound binds to the Huntingtin fibrils.

23. A method of determining the presence of Huntingtin fibrils in a sample obtained from a subject, the method comprising (i) contacting the sample with a peptide agent, wherein the peptide agent comprises one or more peptides comprising an amino acid sequence having at least 70% identity to an amino acid sequence selected from:
(i)
(HD1; SEQ ID NO: 1)
MDWWPMWPSL;
(ii)
(HD2; SEQ ID NO: 2)
MWMIQMPGYQ;
(iii)
(HD3; SEQ ID NO: 3)
MRWSMSYSWA;
(iv)
(HD4; SEQ ID NO: 4)
MFMMMWMSLT;
(v)
(HD5; SEQ ID NO: 5)
MFFVLSWTPL;
(vi)
(HD6; SEQ ID NO: 6)
MQMWTMWEPW;
(vii)
(HD7; SEQ ID NO: 7)
MDLWPMWESW;
(viii)
(HD8; SEQ ID NO: 8)
MWQMMNGMSQ;
(ix)
(HD1-Derivative; SEQ ID NO: 9)
MX₂X₃X₄X₅X₆X₇X₈X₉X₁₀,
wherein X₂ is selected from D, H, L, N, T and Y; X₃ is selected from F, R and W; X₄ is selected from R and W; X₅ is selected from H, P, S and T; X₆ is selected from L, M, Q, S, T and V; X₇ is selected from R and W; X₈ is selected from A, E, H, P, R, S, and T; X₉ is selected from D, P, Q, S, T and W; and X₁₀ is selected from L, M, P, R and T;
(x) MX₂X₃WX₅X₆WX₈X₉X₁₀ (HD6/7-Derivative; SEQ ID NO:10), wherein X₂ is selected from A, D, E, I, L, M, N, P, Q, T and V; X₃ is selected from F, L, M, S, W and Y; X₅ is selected from A, D, E, G, N, P, Q and T; X₆ is selected from F, L, M and W; X₈ is selected from D, E, N, Q and P; X₉ is selected from C, D, N, P, S and T; and X₁₀ is selected from L and W;
(xii) MWX₃X₄X₅X₆X₇X₈X₉Q (HD2-Derivative; SEQ ID NO:11), wherein X₃ is selected from M and Q; X₄ is selected from I and M; X₅ is selected from M and Q; X₆ is selected from F and M; X₇ is selected from I, M, N, P and Q; X₈ is selected from F, G, M, S and W; and X₉ is selected from E and Y; and
(xiii) MX₂QX₄MX₆X₇X₈X₉Q (HD8-Derivative; SEQ ID NO:12), wherein X₂ is selected from F, W and Y; X₄ is selected from L, M, W and Y; X₆ is selected from G, H, N, S and Y; X₇ is selected from G and N; X₈ is selected from I and M; and X₉ is selected from G, S and Y; and
(ii) determining the presence of a complex comprising the peptide agent and a Huntingtin fibril, wherein the presence of said complex indicates the presence of Huntingtin fibrils in the sample.

24. The method of claim 23, wherein the presence of Huntingtin fibrils in the sample indicates that the subject has or is at risk of having Huntington's disease.

25. The method of claim 19, wherein the peptide agent further comprises one or more of the following: a detectable label, one or more amino acid analogues, one or more nucleic acids, a protein tag, a particle, a heavy metal, a cell permeability agent, a carrier or transport protein, a lytic peptide, and/or a half-life enhancing agent.

26. A method of purifying Huntingtin fibrils from a sample obtained from a subject, the method comprising (i) contacting the sample with a peptide agent, wherein the peptide agent comprises one or more peptides comprising an amino acid sequence having at least 70% identity to an amino acid sequence selected from:
(i)
(HD1; SEQ ID NO: 1)
MDWWPMWPSL;
(ii)
(HD2; SEQ ID NO: 2)
MWMIQMPGYQ;
(iii)
(HD3; SEQ ID NO: 3)
MRWSMSYSWA;
(iv)
(HD4; SEQ ID NO: 4)
MFMMMWMSLT;
(v)
(HD5; SEQ ID NO: 5)
MFFVLSWTPL;
(vi)
(HD6; SEQ ID NO: 6)
MQMWTMWEPW;
(vii)
(HD7; SEQ ID NO: 7)
MDLWPMWESW;
(viii)
(HD8; SEQ ID NO: 8)
MWQMMNGMSQ;
(ix)
(HD1-Derivative; SEQ ID NO: 9)
MX₂X₃X₄X₅X₆X₇X₈X₉X₁₀,
wherein X₂ is selected from D, H, L, N, T and Y; X₃ is selected from F, R and W; X₄ is selected from R and W; X₅ is selected from H, P, S and T; X₆ is selected from L, M, Q, S, T and V; X₇ is selected from R and W; X₈ is selected from A, E, H, P, R, S, and T; X₉ is selected from D, P, Q, S, T and W; and X₁₀ is selected from L, M, P, R and T;
(x) MX₂X₃WX₅X₆WX₈X₉X₁₀ (HD6/7-Derivative; SEQ ID NO:10), wherein X₂ is selected from A, D, E, I, L, M, N, P, Q, T and V; X₃ is selected from F, L, M, S, W and Y; X₅ is selected from A, D, E, G, N, P, Q and T; X₆ is selected from F, L, M and W; X₈ is selected from D, E, N, Q and P; X₉ is selected from C, D, N, P, S and T; and X₁₀ is selected from L and W;
(xii) MWX₃X₄X₅X₆X₇X₈X₉Q (HD2-Derivative; SEQ ID NO:11), wherein X₃ is selected from M and Q; X₄ is selected from I and M; X₅ is selected from M and Q; X₆ is selected from F and M; X₇ is selected from I, M, N, P and Q; X₈ is selected from F, G, M, S and W; and X₉ is selected from E and Y; and
(xiii) MX₂QX₄MX₆X₇X₈X₉Q (HD8-Derivative; SEQ ID NO:12), wherein X₂ is selected from F, W and Y; X₄ is selected from L, M, W and Y; X₆ is selected from G, H, N, S and Y; X₇ is selected from G and N; X₈ is selected from I and M; and X₉ is selected from G, S and Y; and (ii) isolating the fibrils from the sample.

27. The method of claim 19, wherein the peptide agent is delivered via a liposome.

28. The peptide agent of claim 1, wherein the one or more peptides have a length of 10 amino acids or less.
